(19) United States
(12) Patent Application Publication
(10) Pub. No.: US 2009/0161100 A1
(43) Pub. Date: Jun. 25, 2009

(54) Fiber Optic Interrogated Microslide, Microslide Kits and Uses Thereof

(76) Inventors: Michael J. Minot, Andover, MA (US); David W. Stowe, Milford, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 12/084,715

(22) PCT Filed: Nov. 8, 2006
(86) PCT No.: PCT/US06/43571

§ 371 Date: May 8, 2008

Related U.S. Application Data
(60) Provisional application No. 60/734,597, filed on Nov. 8, 2005.

(51) Int. Cl. G01N 21/01; B01J 19/00; C12M 1/00; G01N 21/76; C40B 30/10; C12Q 1/70; C12Q 1/02

(52) U.S. Cl. 356/244; 422/61; 435/288.3; 422/52; 506/12; 435/5; 435/29

(57) ABSTRACT

The present invention provides a substrate that overcomes the performance limitations of conventional microscope slides, microarrays, or microtiter plates when optically interrogated through the thickness of the substrate. With conventional microscope slides, image quality and resolution are degraded as a result of distortions introduced by imaging through the thickness of the glass. Fiber Optic Interrogated Microslides (FOI) consist of many fiber optics that have been fused together. When sliced and polished to form microscope slides, the fibers effectively transfer optical images from one surface of the microslide to the other. The finished microslide is the optical equivalent of a zero thickness window. The image of an object on the top surface is transferred to the bottom surface allowing it to be viewed without focusing through the thickness of the slide. In addition to providing improved image quality, FOI microslides allow objects to be directly imaged without complex and expensive focusing optics.

### RELATED APPLICATIONS

This application claims the benefit of U.S. provisional application 60/734,597, filed Nov. 8, 2005. The entire content of that application is hereby incorporated by reference.

### FIELD OF THE INVENTION

The invention relates to a FOI microslide which can be used as a substrate for a microarray, microtiter plate, or other applications such as those involving bottom reading.

### BACKGROUND OF THE INVENTION

The first useful microscope was developed in the Netherlands between 1590 and 1608. For over 400 years of history, microscope slides, typically made of glass, have been used to support the object being studied. With conventional microscopy, a light source at the bottom of the microscope projects light up through a hole in the stage, through the microscope slide and the object being viewed (from above). In an inverted microscope, the light source and condenser are on the top above the stage pointing down. The objectives and turret are below the stage pointing up. The specimen (as dictated by the laws of gravity) is placed on top of the stage. The sample is viewed through the bottom of the slide holding the specimen. Throughout this period of development, the plain microscope slide has remained substantially the same: a clear rectangular homogeneous glass plates used to hold specimens for examination under a microscope.

With an inverted microscope or other bottom reading instruments, the sample is viewed through the thickness of the microscope slide, or through the bottoms of different containers (microtiter plate, for example) with various thicknesses and variable optical characteristics. A standard plain microscope slide is typically 1-2 mm thick. Conventional high power microscope objectives typically have a very short working distance and must get very close to the subject to focus. Because of the finite thickness of a microscope slide, glass bottomed microtiter plate, or the bottom of the container, a standard higher power objective may not be able to get close enough to the subject to focus. Therefore the higher power objectives on an inverted microscope must be corrected for a much longer working distance. Even with all these corrections, the quality of the image may not be as good as looking through a conventional (top down) microscope with comparable objectives. Furthermore, focusing through the thickness of the glass microscope slide, or glass bottomed microtiter plate becomes challenging.

One strategy that is employed to minimize the optical effects associated with viewing through the thickness of a substrate is to produce a very thin substrate. For example, certain glass bottomed microtiter plates are available in which the glass bottom is only 150 microns (0.006″) or less, thick. While minimizing thickness issues, these bottoms lack rigidity creating issues associated with flatness. For example, some plastic microtiter plates are fabricated with glass bottoms. A very thin sheet (150 microns or less) is used to minimize thickness related distortions. Other distortions arise as a result of the fact that the glass and plastic are not expansion matched which cause the glass to warp from one area to another.

Successful completion of the Human Genome Project in 2003 laid the foundation for ongoing development and commercialization of novel technology and instrument systems to enable rapid sequencing of genomes. Utilizing nanotechnology, proprietary chemistry, and novel microfluidic biochips, innovative firms are racing to develop methods and instrument systems that enable diagnostic analysis (sequencing) hundreds of times faster than conventional techniques. A biochip, such as a DNA micro-array, is typically a glass or silicon wafer that is designed for the purpose of accelerating genetic research. It may also be able to rapidly detect chemical agents used in biological warfare so that defensive measures could be taken.

Progress in biological sciences has been accelerated by the advent of microarray technology. In a 2D microarray, biological samples, typically (but not limited to) genomic or proteomic fragments, are deposited or synthesized onto a substrate, in a predetermined spatial order, allowing them to be made available as diagnostic probes in a high-throughput, parallel manner. The substrate is commonly a conventional plain microscope slide, but can also be other materials such as silicon wafer or a filter support matrix. Microarrays allow hundreds and even thousands of reactions to be analyzed on a single plate having the format of a standard microscope slide. For some applications the surface of the microarray might consist of microwells that can be filled with sample.

Various schemes are also used for viewing or reading the microarrays, including conventional microscopy, inverted microscopy, as well as dedicated reading or scanner instruments. Microarray readers are typically either ‘top’ readers or ‘bottom’ readers. Optical information can be directly imaged onto a CCD array (with or without supplemental focusing optics) or can be detected using a laser scanner in conjunction with a photomultiplier detector. In either case, the reader must have clear optical access to the samples on the microarray. Viewing from the top surface allows access under all circumstances but is complicated by the necessary depth of focus of the optics (many millimeters) and by challenges of interrogating the microarray (for example, a droplet of liquid) through liquid. By coming up underneath the plate and passing the light through a transparent base these shortcomings can be negated, however the problems previously described for viewing through a thickness (of a plain microscope slide) become apparent. An issue common to both configurations is that the base of the microarray and the focal plane of the optics have to coincide throughout the scan to produce an optimal signal. One way this can be ensured is by making the base of the substrate flat to a few microns over its entire area. An alternative is to incorporate an active focusing mechanism in the scanner, tracking the height of the scanning beam over the plate to take care of the undulations in the base, and to focus on the target; however the auto-focus optics adds considerably to the cost of the scanner instrument.

### SUMMARY OF THE INVENTION

An embodiment of the present invention provides a substrate material that eliminates the problems (e.g., substrate thicknesses) associated with viewing a conventional plain microscope slide or microarray, or microtiter plate.

Another embodiment of the present invention provides a substrate material that eliminates the adverse optical effects of substrate thickness, allowing the substrate to be fabricated without regard to thickness (1,000 microns—0.039″ or thicker for example), which can eliminate the distortion effects that are typical of thin (150 micron—0.006″) conventional substrates.

Another embodiment provides a microscope slide, microarray or microtiter plate substrate material of the invention that allows direct imaging onto a CCD reader, minimizing the need for costly optics.

Another embodiment of the invention provides a substrate material that offers significantly improved resolution as compared to a conventional microscope slide, microarray substrate or microtiter plate when viewing through the substrate.

Another embodiment of the invention provides a substrate material that offers far greater (e.g., 10,000×) light collection efficiency than conventional microscope slides, microarray substrates or microtiter plates bottoms.

Another embodiment of the invention provides a substrate material that significantly reduces the effects of chromatic dispersion compared to a conventional microscope slide, microarray substrate or microtiter plate.

Another embodiment of the invention provides a substrate material that provides enhanced resolution when viewing an object immersed in a medium having a refractive index greater than air, but less than the index of the substrate material.

Another embodiment provides a substrate material of the invention for microscope slides, microarray substrates or microtiter plates bottoms that incorporates the ability to magnify or reduce the size of the image of the item being viewed (e.g., tapers).

Another embodiment provides a substrate material of the invention that can serve as a bottom viewing microscope slide, microarray substrate, microtiter plate bottom. This embodiment can be used in, for example, applications requiring very thick interrogation plates. Such plates of the invention can offer improved strength, stiffness, rigidity, etc. without requiring complex optics, and without sacrificing resolution.

Another embodiment provides a substrate of the invention that serves as a microscope slide, microarray substrate, microtiter plate bottom that can be provided without any special surface coating (blank microslide) or with a full range of functional coating chemistries for DNA and protein microarraying or other specialty applications.

Another embodiment of the invention provides an integrated kit of components suitable for specialty applications, such as microarraying. For example, a microarraying kit of the invention could include some of the following: one or more microslides, solutions and hardware to deposit microarray samples onto the microslide, reagents for analysis of the microarray, documented procedures for spotting microarrays, and software for analysis of the results.

In one embodiment of the present invention, a Fiber Optic Interrogated Microslide has been developed as a substrate that overcomes the performance limitations of conventional microscope slide, microarray, or microtiter plates. Fiber Optic Interrogated Microslides can comprise many millions of minute fiber optics that have been fused together. When sliced and polished to form plates, the fibers effectively transfer optical images from one surface of the microslide to the other, allowing images on one surface to be viewed on the other, without regard to the thickness of the slide. A microslide of the invention is the optical equivalent of a zero thickness window.

Further exemplary embodiments of the invention are as follows.

1. A fiber optic interrogated microslide capable of zero thickness optical interrogation, the microslide comprising:
a substrate comprising an upper and lower surface; and
a plurality of optic fibers integrally disposed in the substrate, at least one optic fiber optically coupling the upper and lower surfaces of the substrate, wherein optically coupling the upper and lower surfaces of the substrate provides for substantially zero thickness optical interrogation.

2. A fiber optic interrogated microslide capable of zero thickness optical equivalence, the microslide comprising:
a substrate comprising an upper and lower surface;
a sample disposed on the upper or lower surface of the substrate; and
a plurality of optic fibers integrally disposed in the substrate, at least one optic fiber optically coupling the sample and the upper or lower surface of the substrate, wherein optically coupling the sample and the upper or lower surface of the substrate provides for substantially zero thickness optical interrogation.

3. A substrate having a sample disposed on a surface thereof and an imaging device for viewing the sample, wherein the substrate provides for greater light collection efficiency by optically coupling the sample to the imaging device via at least one optic fiber integrally disposed in the substrate.

4. A substrate having a sample disposed on a surface thereof and an imaging device for viewing the sample, wherein the substrate provides for greater resolution by optically coupling the sample to the imaging device via at least one optic fiber integrally disposed in the substrate.

5. A substrate having a sample disposed on a surface thereof and an imaging device for viewing the sample, wherein the substrate reduces chromatic dispersion by optically coupling the sample to the imaging device via at least one optic fiber integrally disposed in the substrate.

6. A substrate having a sample disposed on a surface thereof and an imaging device for viewing the sample, wherein the imaging device is a microscope or charged coupled device reader or camera, the substrate optically coupling the sample to the imaging device via at least one optic fiber integrally disposed in the substrate, wherein the at least one optic fiber provides for substantially zero thickness optical interrogation.

7. A substrate having a sample disposed on a surface thereof and an imaging device for viewing the sample, wherein the substrate is capable of optically coupling the sample to the imaging device via at least one optic fiber integrally disposed in the substrate, wherein the at least one optic fiber provides for substantially zero thickness optical interrogation.

8. A fiber optic interrogated microslide capable of zero thickness optical interrogation, the microslide comprising:
a substrate comprising an upper and lower surface;
an event occurring proximate to the upper or lower surface of the substrate; and
a plurality of optic fibers integrally disposed in the substrate, at least one optic fiber optically coupling the event and the upper or lower surface of the substrate, wherein optically coupling the event and the upper or lower surface of the substrate provides for substantially zero thickness optical interrogation.

9. The microslide of embodiment 6, wherein the event is a biological, chemical or physical event.

10. The microslide of embodiment 6, wherein the event is a chemiluminescent reaction.

11. A fiber optic interrogated microslide capable of zero thickness optical interrogation, the microslide comprising:
a substrate comprising an upper and lower surface;
a plurality of optic fibers integrally disposed in the substrate, at least one optic fiber optically coupling the upper and lower surfaces of the substrate, wherein optically coupling the upper and lower surfaces of the substrate provides for substantially zero thickness optical interrogation; and
an identification member in contact with the substrate.

12. A fiber optic interrogated microslide capable of zero thickness optical interrogation, the microslide comprising:
a substrate comprising an upper and lower surface, wherein the upper, lower or both surfaces of the substrate are passivated; and
a plurality of optic fibers integrally disposed in the substrate, at least one optic fiber optically coupling the upper and lower surfaces of the substrate, wherein optically coupling the upper and lower surfaces of the substrate provides for substantially zero thickness optical interrogation.

13. The microslide of embodiment 12, wherein the upper, lower or both surfaces are passivated by a coating deposited by vacuum evaporation, sputtering, laser ablation, reactive ion platting, plasma enhanced deposition, organo-metallic dipping, spraying or combinations thereof.

14. A kit, the kit comprising:
a substrate comprising an upper and lower surface and a plurality of optic fibers integrally disposed in the substrate; and
a sample to be disposed on the upper or lower surface of the substrate for substantially zero thickness optical interrogation via at least one optic fiber.

15. The kit of embodiment 14, wherein at least one optic fiber is optically coupled to the sample.

16. The kit of embodiment 14, wherein the sample comprises pharmaceutical compounds, genomic components, metallic components, metals, polymeric components, polymers, polyether, ether, ketones, polyimides, epoxies, nylons, homopolymers, heteropolymers, polycarbonates, glass, acetal polymers, acrylate polymers, methacrylate polymers, copolymers, terpolymers, cellulosic polymers, cellulose acetates, cellulose nitrates, cellulose propionates, cellulose acetate butyrates, cellophanes, rayons, rayon triacetates, cellulose ethers, carboxymethyl celluloses, hydroxyalkyl celluloses, polyoxymethylene polymers, polyimide polymers, polyether block imides, polybismaleinimides, polyamidimides, polyesterimides, polyetherimides, polysulfone polymers, polyarylsulfones, polyethersulfones, polyamide polymers, nylon 6,6, polycaprolactams, polyacrylamides, resins, alkyd resins, phenolic resins, urea resins, melamine resins, epoxy resins, allyl resins, epoxide resins, polycarbonates, polyacrylonitriles, polyvinylpyrrolidones, anhydride polymers, maleic anhydride polymers, polymers of vinyl monomers, polyvinyl alcohols, polyvinyl halides, polyvinyl chlorides, ethylene vinylacetate copolymers, polyvinylidene chlorides, polyvinyl ethers, polyvinyl methyl ethers, polystyrenes, styrene butadiene copolymers, acrylonitrile styrene copolymers, acrylonitrile butadiene styrene copolymers, styrene butadiene styrene copolymers, styrene isobutylene styrene copolymers, polyvinyl ketones, polyvinylcarbazoles, polyvinyl esters, polyvinyl acetates, hydrogels, polybenzimidazoles, ionomers, polyalkyl oxide polymers, polyethylene oxides, glycosaminoglycans, polyesters, polyethylene terephthalates, aliphatic polyesters, polymers of lactide, epsilon caprolactone, glycolide, glycolic acid, hydroxybutyrate, hydroxyvalerate, paradioxanone, trimethylene carbonate, 1,4-dioxepan-2-one, 1,5-dioxepan-2-one, 6,6-dimethyl-1,4-dioxan-2-one, polyether polymers, polyarylethers, polyphenylene ethers, polyether ketones, polyether ether ketones, polyphenylene sulfides, polyisocyanates, polyolefin polymers, polyalkylenes, polypropylenes, polyethylenes, polybutylenes, polybut-1-ene, polyisobutylene, poly-4-methyl-pen-1-enes, ethylene-alpha-olefin copolymers, ethylene-methyl methacrylate copolymers, ethylene-vinyl acetate copolymers, fluorinated polymers, polytetrafluoroethylenes, poly(tetrafluoroethylene-co-hexafluoropropene), modified ethylene-tetrafluoroethylene copolymers, polyvinylidene fluorides, silicone polymers, polyurethanes, polyurethane dispersions, p-xylylene polymers, polyiminocarbonates, copoly(ether-esters), polyethylene oxide-polylactic acid copolymers, polyphosphazines, polyalkylene oxalates, polyoxaamides, polyoxaesters, amines, amino groups, polyorthoesters, biopolymers, polypeptides, proteins, polysaccharides, fatty acids, esters of fatty acids, fibrin, fibrinogen, collagen, elastin, chitosan, gelatin, starch, glycosaminoglycans, hyaluronic acid, therapeutic agents, oligonucleotides, proteins, antisense polynucleotides, polynucleotides coding for a specific product, genetic recombinant components, nucleic acids, DNA, cDNA, mRNA, tRNA, RNA, polynucleotides, viruses, bacteria, phage, histones, non-infectious vectors, vectors, plasmids, lipids, liposomes, cationic polymers, cationic lipids, viral vectors, virus-like particles, synthetic virus particles, peptide targeting sequences, antisense nucleic acids, genomic sequences, DNA chimeras, gene sequences encoding for ferry proteins, membrane translocating sequences, cells, ribozymes, antisense oligonucleotides, DNA compacting agents, gene or vector systems, polynucleotides, recombinant nucleic acids, naked DNA, cDNA, mRNA, tRNA or RNA, genomic DNA, cDNA, mRNA, tRNA or RNA in a non-infectious vector or in a viral vector, human origin cells, autologous cells, allogeneic cells, animal source cells, xenogeneic cells, genetically engineered proteins, polymerized chain reaction components, blood, serums, bodily fluids, tissues or any combinations thereof.

17. A kit, the kit comprising:
a substrate comprising an upper and lower surface and a plurality of optic fibers integrally disposed in the substrate; and
a material capable of associating with an event that occurs proximate to the upper or lower surface of the substrate, wherein at least one optic fiber provides for substantially zero thickness optical interrogation of the event.

18. The kit of embodiment 17, wherein at least one optic fiber is optically coupled to the event.

19. A kit, the kit comprising:
a substrate comprising an upper and lower surface and a plurality of optic fibers integrally disposed in the substrate; and
a functional agent for coating the substrate.

20. The kit of embodiment 19, wherein the functional agent comprises aminosilane, epoxy, aldehyde coatings or combinations thereof.

21. The kit of embodiment 16, wherein the functional agent comprises pharmaceutical compounds, genomic components, metallic components, metals, polymeric components, polymers, polyether, ether, ketones, polyimides, epoxies, nylons, homopolymers, heteropolymers, polycarbonates, glass, acetal polymers, acrylate polymers, methacrylate polymers, copolymers, terpolymers, cellulosic polymers, cellulose acetates, cellulose nitrates, cellulose propionates, cellulose acetate butyrates, cellophanes, rayons, rayon triacetates, cellulose ethers, carboxymethyl celluloses, hydroxyalkyl celluloses, polyoxymethylene polymers, polyimide polymers, polyether block imides, polybismaleinimides, polyamidimides, polyesterimides, polyetherimides, polysulfone polymers, polyarylsulfones, polyethersulfones, polyamide polymers, nylon 6,6, polycaprolactams, polyacrylamides, resins, alkyd resins, phenolic resins, urea resins, melamine resins, epoxy resins, allyl resins, epoxide resins, polycarbonates, polyacrylonitriles, polyvinylpyrrolidones, anhydride polymers, maleic anhydride polymers, polymers of vinyl monomers, polyvinyl alcohols, polyvinyl halides, polyvinyl chlorides, ethylene vinylacetate copolymers, polyvinylidene chlorides, polyvinyl ethers, polyvinyl methyl ethers, polystyrenes, styrene butadiene copolymers, acrylonitrile styrene copolymers, acrylonitrile butadiene styrene copolymers, styrene butadiene styrene copolymers, styrene isobutylene styrene copolymers, polyvinyl ketones, polyvinylcarbazoles, polyvinyl esters, polyvinyl acetates, hydrogels, polybenzimidazoles, ionomers, polyalkyl oxide polymers, polyethylene oxides, glycosaminoglycans, polyesters, polyethylene terephthalates, aliphatic polyesters, polymers of lactide, epsilon caprolactone, glycolide, glycolic acid, hydroxybutyrate, hydroxyvalerate, paradioxanone, trimethylene carbonate, 1,4-dioxepan-2-one, 1,5-dioxepan-2-one, 6,6-dimethyl-1,4-dioxan-2-one, polyether polymers, polyarylethers, polyphenylene ethers, polyether ketones, polyether ether ketones, polyphenylene sulfides, polyisocyanates, polyolefin polymers, polyalkylenes, polypropylenes, polyethylenes, polybutylenes, polybut-1-ene, polyisobutylene, poly-4-methyl-pen-1-enes, ethylene-alpha-olefin copolymers, ethylene-methyl methacrylate copolymers, ethylene-vinyl acetate copolymers, fluorinated polymers, polytetrafluoroethylenes, poly(tetrafluoroethylene-co-hexafluoropropene), modified ethylene-tetrafluoroethylene copolymers, polyvinylidene fluorides, silicone polymers, polyurethanes, polyurethane dispersions, p-xylylene polymers, polyiminocarbonates, copoly(ether-esters), polyethylene oxide-polylactic acid copolymers, polyphosphazines, polyalkylene oxalates, polyoxaamides, polyoxaesters, amines, amino groups, polyorthoesters, biopolymers, polypeptides, proteins, polysaccharides, fatty acids, esters of fatty acids, fibrin, fibrinogen, collagen, elastin, chitosan, gelatin, starch, glycosaminoglycans, hyaluronic acid, therapeutic agents, oligonucleotides, proteins, antisense polynucleotides, polynucleotides coding for a specific product, genetic recombinant components, nucleic acids, DNA, cDNA, mRNA, tRNA, RNA, polynucleotides, viruses, bacteria, phage, histones, non-infectious vectors, vectors, plasmids, lipids, liposomes, cationic polymers, cationic lipids, viral vectors, virus-like particles, synthetic virus particles, peptide targeting sequences, antisense nucleic acids, genomic sequences, DNA chimeras, gene sequences encoding for ferry proteins, membrane translocating sequences, cells, ribozymes, antisense oligonucleotides, DNA compacting agents, gene or vector systems, polynucleotides, recombinant nucleic acids, naked DNA, cDNA, mRNA, tRNA or RNA, genomic DNA, cDNA, mRNA, tRNA or RNA in a non-infectious vector or in a viral vector, human origin cells, autologous cells, allogeneic cells, animal source cells, xenogeneic cells, genetically engineered proteins, polymerized chain reaction components, blood, serums, bodily fluids, tissues or any combinations thereof.

22. A method for zero thickness optical interrogation, the method comprising:
providing a substrate comprising an upper and lower surface and a plurality of optic fibers integrally disposed in the substrate; and
optically coupling the upper and lower surfaces of the substrate via at least one optic fiber for substantially zero thickness optical interrogation.

23. A method for zero thickness optical interrogation of a sample, the method comprising:
providing a substrate comprising an upper and lower surface and a plurality of optic fibers integrally disposed in the substrate;
disposing a sample on the upper or lower surface of the substrate; and
optically coupling the sample and the upper or lower surface of the substrate via at least one optic fiber for substantially zero thickness optical interrogation.

24. A method for zero thickness optical interrogation of an event, the method comprising:
providing a substrate comprising an upper and lower surface and a plurality of optic fibers integrally disposed in the substrate;
initiating an event proximate to the upper or lower surface of the substrate; and

optically coupling the event and the upper or lower surface of the substrate via at least one optic fiber for substantially zero thickness optical interrogation.

25. A method for zero thickness optical interrogation, the method comprising:
providing a substrate comprising an upper and lower surface and a plurality of optic fibers integrally disposed in the substrate;
disposing a sample on or initiating an event proximate to the upper or lower surface of the substrate; and

optically coupling the sample or event to an imaging device via at least one optic fiber for substantially zero thickness optical interrogation.

26. The method of embodiment 25, wherein the imaging device is a microscope or charged couple device reader or camera.

27. A method for zero thickness optical interrogation, the method comprising:

providing a substrate comprising an upper and lower surface and a plurality of optic fibers integrally disposed in the substrate; and

using the substrate as a microtiter plate, microscope slides, microarray plate or combinations thereof.

28. A method comprising:

providing a substrate comprising an upper and lower surface and a plurality of optic fibers integrally disposed in the substrate; and

partially, substantially or completely coating the upper, lower or both surfaces of the substrate with a functional agent.

29. The method of embodiment 28, wherein the functional agent comprises aminosilane, epoxy, aldehyde coatings or combinations thereof.

30. The method of embodiment 24, wherein the functional agent comprises pharmaceutical compounds, genomic components, metallic components, metals, polymeric components, polymers, polyether, ether, ketones, polyimides, epoxies, nylons, homopolymers, heteropolymers, polycarbonates, glass, acetal polymers, acrylate polymers, methacrylate polymers, copolymers, terpolymers, cellulosic polymers, cellulose acetates, cellulose nitrates, cellulose propionates, cellulose acetate butyrates, cellophanes, rayons, rayon triacetates, cellulose ethers, carboxymethyl celluloses, hydroxyalkyl celluloses, polyoxymethylene polymers, polyimide polymers, polyether block imides, polybismaleinimides, polyamidimides, polyesterimides, polyetherimides, polysulfone polymers, polyarylsulfones, polyethersulfones, polyamide polymers, nylon 6,6, polycaprolactams, polyacrylamides, resins, alkyd resins, phenolic resins, urea resins, melamine resins, epoxy resins, allyl resins, epoxide resins, polycarbonates, polyacrylonitriles, polyvinylpyrrolidones, anhydride polymers, maleic anhydride polymers, polymers of vinyl monomers, polyvinyl alcohols, polyvinyl halides, polyvinyl chlorides, ethylene vinylacetate copolymers, polyvinylidene chlorides, polyvinyl ethers, polyvinyl methyl ethers, polystyrenes, styrene butadiene copolymers, acrylonitrile styrene copolymers, acrylonitrile butadiene styrene copolymers, styrene butadiene styrene copolymers, styrene isobutylene styrene copolymers, polyvinyl ketones, polyvinylcarbazoles, polyvinyl esters, polyvinyl acetates, hydrogels, polybenzimidazoles, ionomers, polyalkyl oxide polymers, polyethylene oxides, glycosaminoglycans, polyesters, polyethylene terephthalates, aliphatic polyesters, polymers of lactide, epsilon caprolactone, glycolide, glycolic acid, hydroxybutyrate, hydroxyvalerate, paradioxanone, trimethylene carbonate, 1,4-dioxepan-2-one, 1,5-dioxepan-2-one, 6,6-dimethyl-1,4-dioxan-2-one, polyether polymers, polyarylethers, polyphenylene ethers, polyether ketones, polyether ether ketones, polyphenylene sulfides, polyisocyanates, polyolefin polymers, polyalkylenes, polypropylenes, polyethylenes, polybutylenes, polybut-1-ene, polyisobutylene, poly-4-methyl-pen-1-enes, ethylene-alpha-olefin copolymers, ethylene-methyl methacrylate copolymers, ethylene-vinyl acetate copolymers, fluorinated polymers, polytetrafluoroethylenes, poly(tetrafluoroethylene-co-hexafluoropropene), modified ethylene-tetrafluoroethylene copolymers, polyvinylidene fluorides, silicone polymers, polyurethanes, polyurethane dispersions, p-xylylene polymers, polyiminocarbonates, copoly(ether-esters), polyethylene oxide-polylactic acid copolymers, polyphosphazines, polyalkylene oxalates, polyoxaamides, polyoxaesters, amines, amino groups, polyorthoesters, biopolymers, polypeptides, proteins, polysaccharides, fatty acids, esters of fatty acids, fibrin, fibrinogen, collagen, elastin, chitosan, gelatin, starch, glycosaminoglycans, hyaluronic acid, therapeutic agents, oligonucleotides, proteins, antisense polynucleotides, polynucleotides coding for a specific product, genetic recombinant components, nucleic acids, DNA, cDNA, mRNA, tRNA, RNA, polynucleotides, viruses, bacteria, phage, histones, non-infectious vectors, vectors, plasmids, lipids, liposomes, cationic polymers, cationic lipids, viral vectors, virus-like particles, synthetic virus particles, peptide targeting sequences, antisense nucleic acids, genomic sequences, DNA chimeras, gene sequences encoding for ferry proteins, membrane translocating sequences, cells, ribozymes, antisense oligonucleotides, DNA compacting agents, gene or vector systems, polynucleotides, recombinant nucleic acids, naked DNA, cDNA, mRNA, tRNA or RNA, genomic DNA, cDNA, mRNA, tRNA or RNA in a non-infectious vector or in a viral vector, human origin cells, autologous cells, allogeneic cells, animal source cells, xenogeneic cells, genetically engineered proteins, polymerized chain reaction components, blood, serums, bodily fluids, tissues or any combinations thereof.

31. A method comprising:

providing a substrate comprising an upper and lower surface and a plurality of optic fibers integrally disposed in the substrate;

partially, substantially or completely coating the upper, lower or both surfaces of the substrate with a functional agent; and

partially, substantially or completely immobilizing at least a portion of a material via the functional agent.

32. The method of embodiment 31, wherein the material comprises a biological, synthetic, metallic compound or combinations thereof.

33. The method of embodiment 31, wherein the material comprises pharmaceutical compounds, genomic components, metallic components, metals, polymeric components, polymers, polyether, ether, ketones, polyimides, epoxies, nylons, homopolymers, heteropolymers, polycarbonates, glass, acetal polymers, acrylate polymers, methacrylate polymers, copolymers, terpolymers, cellulosic polymers, cellulose acetates, cellulose nitrates, cellulose propionates, cellulose acetate butyrates, cellophanes, rayons, rayon triacetates, cellulose ethers, carboxymethyl celluloses, hydroxyalkyl celluloses, polyoxymethylene polymers, polyimide polymers, polyether block imides, polybismaleinimides, polyamidimides, polyesterimides, polyetherimides, polysulfone polymers, polyarylsulfones, polyethersulfones, polyamide polymers, nylon 6,6, polycaprolactams, polyacrylamides, resins, alkyd resins, phenolic resins, urea resins, melamine resins, epoxy resins, allyl resins, epoxide resins, polycarbonates, polyacrylonitriles, polyvinylpyrrolidones, anhydride polymers, maleic anhydride polymers, polymers of vinyl monomers, polyvinyl alcohols, polyvinyl halides, polyvinyl chlorides, ethylene vinylacetate copolymers, polyvinylidene chlorides, polyvinyl ethers, polyvinyl methyl ethers, polystyrenes, styrene butadiene copolymers, acrylonitrile styrene copolymers, acrylonitrile butadiene styrene copolymers, styrene butadiene styrene copolymers, styrene isobutylene styrene copolymers, polyvinyl ketones, polyvinylcarbazoles, polyvinyl esters, polyvinyl acetates, hydrogels, polybenzimidazoles, ionomers, polyalkyl oxide polymers, polyethylene oxides, glycosaminoglycans, polyesters, polyethylene terephthalates, aliphatic polyesters, polymers of lactide, epsilon caprolactone, glycolide, glycolic acid, hydroxybutyrate, hydroxyvalerate, paradioxanone, trimethylene carbonate, 1,4-dioxepan-2-one, 1,5-dioxepan-2-one, 6,6-dimethyl-1,4-dioxan-2-one, polyether polymers, polyarylethers, polyphenylene ethers, polyether ketones, polyether ether ketones, polyphenylene sulfides, polyisocyanates, polyolefin polymers, polyalkylenes, polypropylenes, polyethylenes, polybutylenes, polybut-1-ene, polyisobutylene, poly-4-methyl-pen-1-enes, ethylene-alpha-olefin copolymers, ethylene-methyl methacrylate copolymers, ethylene-vinyl acetate copolymers, fluorinated polymers, polytetrafluoroethylenes, poly(tetrafluoroethylene-co-hexafluoropropene), modified ethylene-tetrafluoroethylene copolymers, polyvinylidene fluorides, silicone polymers, polyurethanes, polyurethane dispersions, p-xylylene polymers, polyiminocarbonates, copoly(ether-esters), polyethylene oxide-polylactic acid copolymers, polyphosphazines, polyalkylene oxalates, polyoxaamides, polyoxaesters, amines, amino groups, polyorthoesters, biopolymers, polypeptides, proteins, polysaccharides, fatty acids, esters of fatty acids, fibrin, fibrinogen, collagen, elastin, chitosan, gelatin, starch, glycosaminoglycans, hyaluronic acid, therapeutic agents, oligonucleotides, proteins, antisense polynucleotides, polynucleotides coding for a specific product, genetic recombinant components, nucleic acids, DNA, cDNA, mRNA, tRNA, RNA, polynucleotides, viruses, bacteria, phage, histones, non-infectious vectors, vectors, plasmids, lipids, liposomes, cationic polymers, cationic lipids, viral vectors, virus-like particles, synthetic virus particles, peptide targeting sequences, antisense nucleic acids, genomic sequences, DNA chimeras, gene sequences encoding for ferry proteins, membrane translocating sequences, cells, ribozymes, antisense oligonucleotides, DNA compacting agents, gene or vector systems, polynucleotides, recombinant nucleic acids, naked DNA, cDNA, mRNA, tRNA or RNA, genomic DNA, cDNA, mRNA, tRNA or RNA in a non-infectious vector or in a viral vector, human origin cells, autologous cells, allogeneic cells, animal source cells, xenogeneic cells, genetically engineered proteins, polymerized chain reaction components, blood, serums, bodily fluids, tissues or any combinations thereof.

34. The method of embodiment 33, wherein immobilization is through covalent bonding, chemical interactions, physical interactions, electrostatic interactions, mechanical interactions, hybridization and combinations thereof.

35. A method comprising:

providing a substrate comprising an upper and lower surface and a plurality of optic fibers integrally disposed in the substrate; and

forming a microarray on the upper or lower surface of the substrate.

36. The method of embodiment 35, wherein the microarray is formed by split pin printing, spotting or combinations thereof.

37. A method, the method comprising:
providing a substrate comprising an upper and lower surface and a plurality of optic fibers integrally disposed in the substrate; and
using at least one optic fiber of the substrate for gene expression monitoring, mutation detection, mutation analyzing, genotyping, genomic mapping, clone mapping, protein detecting, protein quantifying, assaying, microarraying or any combination thereof.

### BRIEF DESCRIPTION OF THE DRAWINGS

Other features and advantages of the present invention will be apparent from the detailed description of the invention that follows, taken in conjunction with the accompanying drawings of which:

FIG. 1 shows three common scanning modes used in laser readers;

FIG. 2 shows a commonly used scheme for top down inspection of a microarray substrate and/or embodiment of the invention; A laser beam is used to excite a fluorescent reaction, which is detected through a focusing objective lens;

FIG. 3 shows the use of CCD camera for top down scanning of microarray substrates and/or embodiments (e.g., a FOI microslide) of the invention;

FIG. 4 shows an approach used for bottom scanning of microarray plates and/or embodiments (e.g., a FOI microslide) of the invention with a high speed galvonometer mirror;

FIG. 5 (A) depicts a microtiter plate being optically interrogated by a single lens that focuses through the plate bottom; (B) shows imaging of whole plate from above; (C) shows a scenario in which multiple microlenses are used to focus through the bottom thickness of the microtiter (or microarray) plate;

FIG. 6 depicts a large format fiber-optic taper-coupled CCD camera; The light sensitive sensor is housed in the white box; The black cone shaped portion is a fiber optic taper; The fiber optic taper is 200-mm in diameter; It is an integral part of the camera, and is bonded directly to the CCD chip;

FIG. 7 shows one embodiment of the invention, a microarray FOI microslide, with optional samples spotted onto the surface being inserted into a sample holding fixture which will hold the microslide bottom in direct contact with the camera faceplate (below); The microslide bottom is placed directly over and in direct contact with the faceplate of the CCD camera; Light producing reactions (fluorescent or luminescent) occurring on the surface of the microarray microslide substrate of the invention are simultaneously monitored through the substrate bottom by the CCD camera;

FIG. 8a is a partial representation of a FOI microslide substrate of the invention integrally composed of a plurality of optic fibers; FIG. 8b represents an embodiment having two FOI microslides stacked one upon the other, with optic fibers in register, and with a surface of the first substrate adjacent to and contacting a surface of the second substrate.

FIG. 9 is a representation of one approach for manufacturing FOI microslides of the invention;

FIG. 10 shows a conventional microscope slide, having a thickness ‘T’; A sensor or detector in contact with the bottom of the slide is depicted (green) below the slide; Such a sensor or detector can be used with an embodiment of the invention;

FIG. 11 shows the effect of chromatic dispersion with conventional microscope slides; The refractive index of many glasses varies depending on the wavelength of light; Differences in the refraction for different wavelengths causes distorted images with conventional microscope slides, sometimes seen as a halo effect;

FIG. 12 depicts the optical performance of a Fiber Optic Interrogated Microslide;

FIG. 13 illustrates the capture of radiation from an isotropic point source for a fiber (such as used for an embodiment of the invention) with NA<1 (acceptance angle less than 90°);

FIG. 14 depicts a scenario in which the light source (object being detected) is suspended in a medium (air, liquid; etc.) above the surface of the FOI Microslide;

FIG. 15 shows how the FOI Microslide collects and then transmits light emanating from a point source illuminating the acceptance circle of radius R;

FIG. 16 depicts an isotropic point source of total optical power Pₒ located a height ‘d’ above the Microslide of the invention. A second source separated a lateral distance ‘x’ from the first one is also shown;

FIG. 17 shown in plot on the left that even for an isotropic source above a microslide of the invention with NA=1, the received optical power per fiber has a maximum directly beneath the source and then decreases as the angle of the fiber from the source increases; The plot on the right shows the resolution achieved for the case where the source elevation is twice the fiber core diameter and the source separation is 5 times the fiber core diameter; It was found that two distinct peaks are observed (resolved) if the source separation is about 2.5 times greater than the elevation above the microslide of the invention;

FIG. 18 shows the use of split pin printing to enable high-speed manufacture of microarrays on Microslides and other surfaces of the invention; Split pins work on the same principal as vintage dip pen tips used for writing; The split pins have flat tips and defined uptake channels, which allows a thin (25 μm) layer of liquid sample to form at the end of the pin, and printing to proceed by gentle surface contact; Split pin printing occurs as a simple 3-step “ink-stamping” process as follows: (a) downstroke, (b) contact, and (c) upstroke; Pin tips and channels are available in a wide assortment of dimensions, allowing users to specify spot diameter and the number of spots per loading;

FIG. 19 Shows a microslide with a droplet spotted onto the surface; The droplet is applied by microarray spotting techniques, and is large enough to conduct biological studies within the droplet; Fluorescent or luminescent reactions within the spot are optically monitored via one or many microslide fibers interrogating each droplet;

FIG. 20 illustrates the effect of fill fluid on NA, acceptance angle and resolution; The figure illustrates the case for a Microslide with a calculated NA=1.010; In air, all of the light emitted downward at angles of 90° or less (gray and yellow areas) is transmitted by the recipient fibers; When a fill fluid with index of 1.33 is added, the acceptance angle falls to 49°; All of the light emitted into the gray area is no longer captured by the outlying fibers, but all of the light emitted into the yellow continues to be captured and transmitted; Therefore, the addition of the fluid can prevent the outlying fibers from transmitting light and degrading the system resolution;

FIG. 21 depicts one embodiment of the invention comprising various multifunctional aminosilane coatings disposed on the surface of a microslide; These coatings, for example, enhance electrostatic attraction and provide improved binding and immobilization of cDNA molecules and PCR products;

FIG. 22 depicts one embodiment of the invention comprising epoxy coatings used to enhance the surface of the microslide for covalent immobilization of amino-modified and unmodified oligonucleotides; The nucleic acids react with the epoxy modified surface to form a stable covalent bond; and

FIG. 23 depicts one embodiment of the invention comprising aldehyde-group coatings used to enhance the surface of the Microslide for covalent immobilization of amino-modified nucleic acids or small protein fragments such as peptides. The invention also contemplates any type of coating applied to a Microslide.

### DETAILED DESCRIPTION OF THE INVENTION

A fiber optic interrogated (FOI) microslide of the invention includes a substrate having an upper surface and a lower surface. The substrate contains a plurality of optic fibers that are integrally disposed within the substrate and that optically couple the upper and lower surfaces of the substrate so as to provide substantially zero thickness optical interrogation of an object or sample resting upon or near to the upper or lower surface of the substrate.

In some embodiments, the optic fibers are essentially parallel to one another (i.e., their longitudinal axes are within 10 degrees of one another, and in certain embodiments within one degree of one another). The optic fibers can be arranged so that their long axis is essentially normal to either the upper or lower surface of the substrate, or both. However, other arrangements of optic fibers are possible. For example, a microslide of the invention can function as a taper or inverter, in which case the substrate will contain at least some regions where the optic fibers are curved with respect to one another.

The upper and lower surfaces of the substrate of a FOI microslide can be, but need not be, substantially parallel. In straight read-through applications such as microscopy, where the microslide serves to image an object on its upper surface, or where the microslide serves as the faceplate for a CCD camera, the upper and lower surfaces of the substrate most often will be substantially parallel, meaning that the planes defined by the upper and lower surfaces will be parallel to within ten degrees, and in certain embodiments to within one degree or less. In other embodiments the upper and lower surfaces of the substrate are not parallel. For example, the light from interrogation at one surface of the substrate may be transmitted at an angle different from 180 degrees to the other surface, where the light is received and processed or analyzed, according to the geometric requirements of a given device or instrumentation set up.

FOI microslides of the invention are capable of providing substantially zero thickness optical interrogation. That is, FOI microslides of the invention produce substantially reduced optical distortion, loss of light intensity due to spreading, or chromatic dispersion of light traveling from one surface of the microslide to another along the optic fiber path when compared with a conventional glass plate. This means that the amount of detectable distortion, loss of intensity, or chromatic dispersion, if any, is essentially independent of the optic fiber path length, i.e., the thickness of the microslide. For example, increasing the thickness of a microslide from 1 mm to 1 cm would result in substantially reduced distortion, loss of intensity due to spreading, or chromatic dispersion when compared to the performance of a conventional glass plate in a contact imaging application. FOI microslides act as zero thickness substrates transmitting optical signals from top to bottom without spreading, so that fluorescent or luminescent activity on the surface can be directly coupled to a CCD device without additional optics. This process is also referred to as “image plane transfer.”

Samples that can be interrogated via a microslide of the invention include, without limitation, molecular, cellular, proteomic, or genomic materials or assays. Any biological, chemical or physical event associated with such materials or assays can also be interrogated by a microslide of the invention. The optic fibers of a microslide enable it to be used for simultaneously interrogating multiple samples or events.

The optic fibers of a microslide of the invention can be coupled to standard detection equipment including, for example, at least one charged coupled device (CCD) or other sensor array device, film camera, microscope, spectrophotometer, fluorometer, or photodetector. Conventional detection equipment often comprises hardware and software appropriate for optical interrogation.

The term “interrogation” can refer to any observation, analysis or examination of a sample. An observation, analysis or examination may be facilitated by using at least one optic fiber or a related element such as an optical fiber probe. An observation, analysis or examination could further be aided by conventional detection equipment such as, for example, a charged coupled device (CCD) or an automated auto-focusing microscope.

The term “microarray” or “microarray plate” generally refers to a plate comprising an array of features. The plate can, for example, comprise a plurality of uniformly distributed wells with each well being from about 1 micrometer (↑m) to 250 ↑m in diameter and capable of physically containing or holding a sample such as a material or an assay.

The term “tolerance” or “glass tolerance” generally relates to a limited or reduced optical resolution, sensitivity and so forth that results when an observation, analysis, interrogation or examination is performed or carried out through the thickness of a substantially translucent material without optic fibers, such as, for instance, glass, through which an observation, analysis, interrogation or examination is carried out or performed. For example, a glass cover slide from beneath which an optical interrogation is performed has an inherent glass tolerance that increases with the thickness of the glass. A microslide of the invention does may not have an inherent tolerance and can provide for a zero thickness optical interrogation. For example, a microslide of the invention can transmit light from one surface thereof to another microslide surface by allowing light to pass through at least one optic fiber or optical fiber probe, which may not limit or reduce optical resolution, sensitivity and so forth that could result when an observation, analysis, interrogation or examination is performed or carried out.

The term “event” can generally refers to a type of biological, chemical or physical occurrence or activity such as observed in, without limitation, molecular, cellular, proteomic, genomic, gaseous materials or assays and any combinations thereof. Such biological, chemical or physical occurrences or activities can include, but are not limited to, reactions, chemiluminescence, mitosis, fluorescence, degradation or growth.

A microslide of the invention is particularly well suited to be used with at least one auto-focusing microscope as it may not have an intrinsic glass tolerance. Absent an intrinsic glass tolerance, any sort of interrogation need not be performed through a glass or plastic cover slip or any other translucent platform without optic fibers. For a conventional auto-focusing microscope, such cover slides or other translucent platforms without optic fibers can act as windows through which an interrogation or observation is made. An observation made through a window poses significant focusing problems for such microscopes. An exemplary microslide of the invention may overcome any focusing related issues by not requiring a glass or plastic cover slip or any other translucent platform without optic fibers through which an interrogation is made from above or below. Without such a glass or plastic cover slip or translucent platform, the quality, resolution and rate of sample interrogation, among other things, tends to improve. Cook et al., “Fiberoptics for displays,” Inf. Disp., pp. 14-16 (1991).

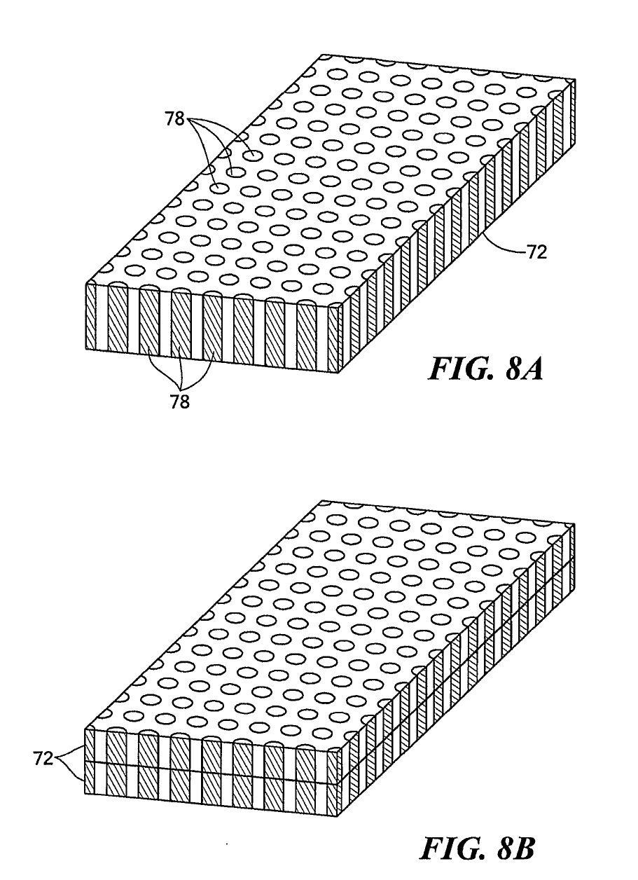

FIG. 8 also shows that the optic fibers comprise a core glass region surrounded by clad glass. The optic fibers are oriented to receive light through an upper end and emit light through a lower end for detection using conventional detection equipment. An example of such detection equipment may include hardware and software that is suitable for optical interrogation. The fibers can be oriented to extend from an upper surface of the microslide to a lower surface thereof. The fibers can also be oriented to extend longitudinally from an upper surface of the microslide to a lower surface thereof. For example, a longitudinal axis of each fiber can pass through the upper and lower surfaces of the microslide. Preferably, the fibers can be orientated orthogonal to the upper and lower surfaces of the microslide. An embodiment of the invention may comprise standard detection equipment including at least one CCD. The CCD can be coupled to at least one optic fiber to receive data or a signal from the fiber and electronically convert such to an image. The image that is obtained represents an interrogation performed by at least one optic fiber from the plurality that comprises the microslide. An example of the components, materials and assemblies of standard detection equipment and at least one CCD coupled to an optic fiber is described by Schempp et al., “Large area CCD-fiber optic imager assembly,” Proc. SPIE, Vol. 1901, pp. 142-45 (1993).

The microslide of the invention could further be comprised of any suitable material. The materials might include those that do not interfere with optical interrogation and permit good adhesion of a deposited sample or functionalizing agent. The microslide may also integrally comprise optic fibers surrounded by a biological, biopolymer synthetic, metallic, polymer or nonmetal material. For example, the microslide could integrally comprise optic fibers set in a matrix of plastic. As discussed above, the optic fibers can have a central core glass region surrounded by fused clad glass. In a preferred embodiment, the core glass may comprise a central region of each optic fiber comprising the microslide.

A microslide of the invention can also comprise optic fibers that are partially or entirely plastic as well as those that have a hollow core region. In an embodiment according to the invention, the microslide could integrally comprise many types of interrogation or diagnostic elements. These elements can further include fiber optic probes or any device that is microsized and capable of interrogating or analyzing molecular, cellular, proteomic, genomic or gaseous materials or assays and any biological, chemical or physical event associated with such materials or assays that occurs along or near the surface of the microslide. Such diagnostic elements may refer to optic fibers that are substantially glass or plastic.

Conventional manufacturing practices can been used for fabricating a microslide such as shown within FIG. 8. A typical manufacturing process can begin with a core glass rod sized to fit within a clad glass tube. The core glass rod and clad glass tube are then loaded into a furnace in which the rod and the tube are fused together and drawn into a length of cane having a standard diameter of approximately 2.5 millimeters (mm). Several lengths of cane are assembled into billets that can be redrawn to yield a multi-structure. The multi-structure may then be assembled into a second billet that is also drawn to form a multiple multi-structure. These billet multi-structures are then cut into a desired length and stacked into a pressing fixture forming an assembled mold. The assembled mold is then placed in a pressing furnace.

The pressing furnace heats and softens the cane lengths as a load is applied to the mold. The resulting block is then annealed and fabricated into a microslide integrally comprising core glass optic fibers surrounded by fused clad glass. The microslide can be cut into rectangular plates having a nominal thickness intended for a specific application or use. The microslide might then be ground and polished to particular dimensions using one or more glass finishing slurries and pad materials. Other variations and modifications relating to the fabrication of a microslide according to the invention might be apparent to a person of ordinary skill in the art.

The fabrication of a microslide can be performed in the generally manner described by Krans, “An introduction to fiber optic imaging,” 1st edition, Schott Fiber Optics, Incorporated. The reference further describes detection equipment such as a CCD that could be used with the optic fibers of the invention for interrogation. The reference also describes, among other things, arrangements, configurations, assemblies, materials or any other variations for optic fibers that may be used to integrally comprise a microslide. Microslide fabrication according to the invention is further disclosed and described within U.S. Pat. Nos. 4,778,501 and 4,925,473, which are incorporated by reference herein.

A microslide of the invention can be fabricated by such described conventional manufacturing practices. A microslide fabricated by these standard practices is generally comprised of optic fibers arranged and aligned with one another such that the axes of the optic fibers are perpendicular to the light input and output surfaces of the microslide. As mentioned, such a microslide does not have an intrinsic tolerance as light impinging on the input surface is directly transmitted to the output surface. This result tends to limit the extent of any optical distortion and can also improve interrogation resolution. A microslide according to the invention might also comprise optic fibers that are tapered for more efficient light collection. A microslide of the invention further does not require that fiber optic interrogation occur through, for instance, a glass or plastic cover slide or any other translucent platform without optic fibers. These slides or platforms act as windows through which light is gathered and transmitted and can effect optical resolution and quality as well as increase the extent of optical distortion.

The selection of both clad and core glass for the optic fibers of a microslide is accomplished such that their chemical and physical properties can be matched. The ratio of core glass area to the total area of an optic fiber may vary depending on a particular application. A typical percentage of core glass area to total area is approximately 70 percent (%) to 90%. The optical properties of a given optic fiber similarly depend on a relative refractive index between the core and clad glass. In one type of embodiment, it may be preferable for the refractive index of the core glass to be larger than that of the clad glass such that incident light will be constrained to the core of the optic fiber and not leak into the clad glass.

The invention further contemplates use of an extra mural absorption (EMA) glass. EMA glass is a type of glass that is highly absorptive and can be integral with the clad glass of a microslide to absorb light that leaks from the core glass of an optic fiber. The absorption of light leaking from the core glass of an optic fiber tends to improve optical signaling and image quality. As described, optic fibers that integrally comprise a microslide of the invention can be substantially (e.g., not horizontal) orthogonal or perpendicular to the surface of the microslide. Substantially orthogonal or perpendicular to the surfaces of a microslide can also describe a fiber that does not pass from one end of the microslide to the other, but rather communicates with the surfaces (e.g., upper and lower) of the microslide. The invention also contemplates other optic fiber configurations that could be used to enhance any sort of interrogation.

A microslide of the invention minimizes or prevents light loss and optical distortion that can occur during interrogation. A microslide integrally comprising optic fibers also has superior resolution and optical transmission in comparison to typical systems for interrogation that observe biological, chemical and physical events through, for example, a glass or plastic window. In one embodiment, the fibers provide an optical link between the interrogated microslide surface and standard detection equipment such as at least one CCD. This arrangement can overcome common interrogation problems or limitations such as encountered when standard detection equipment is used for direct observation or when interrogation occurs through an optical lens or translucent platform without optic fibers. The use of at least one CCD for direct observation, for example, generally requires that the interrogated surface be flat. Interrogation via optic fibers of the invention coupled to at least one CCD does not require a flat surface for interrogation.

Microslides according to the invention and their optic fibers can also comprise, but are not limited to, any of the arrangements, configurations, assemblies, materials or any other variations disclosed and described within U.S. Pat. Nos. 4,693,552, 4,669,813, 4,647,152, 4,591,232 and 4,533,210, which are incorporated by reference herein. Each of the literature, patent or published application references referred to in the present application are also incorporated by reference herein.

The present invention further provides a method for interrogating multiple samples in parallel or individually via the microslides fabricated according to the invention. Such interrogations may include any of those that are described above or other interrogations, analyses or diagnostics that could be contemplated.

The embodiments described above can be used as in either conventional microarray or microtiter plate applications. A microslide according to the invention could further be used for an application that typically would involve a biosensor or biochip employing special loading features and amplification chambers. In one embodiment, a microslide could be used to detect small changes in a specific deoxyribonucleic acid (DNA) sequence. The microslide could further be used to detect a single nucleotide polymorphism (SNP), which might indicate a predisposition to a disease. A microslide of the invention may also be used as a platform or structure for polymerase chain reaction (PCR) amplification.

In another embodiment, the microslide could also be used in conjunction with array technology to enable both cost effective and high-throughput genotyping. Similar array type technology used in conjunction with a microslide of the invention may be effective for recombinant nucleic acid (RNA) profiling. Bacteria, viruses, cells and so forth can also be grown and monitored by a disclosed microslide. A microslide of the invention could further be fabricated such that conventional automated equipment may deposit samples onto or withdraw samples from the microslide.

The method and microslide of the invention can also be used to conveniently investigate up to, without limitation, thousands of material or assay samples in parallel or individually. These material or assay samples could include, for instance, various molecular, cellular, proteomic, genomic or gaseous materials or assays. A microslide according to the invention can also be used to interrogate sample reagents flowed across the microslide or affixed thereto using, for example, a functionalizing agent. The embodiments according to the invention tend to enhance optical interrogation resolution when compared to other optical-based processes or techniques.

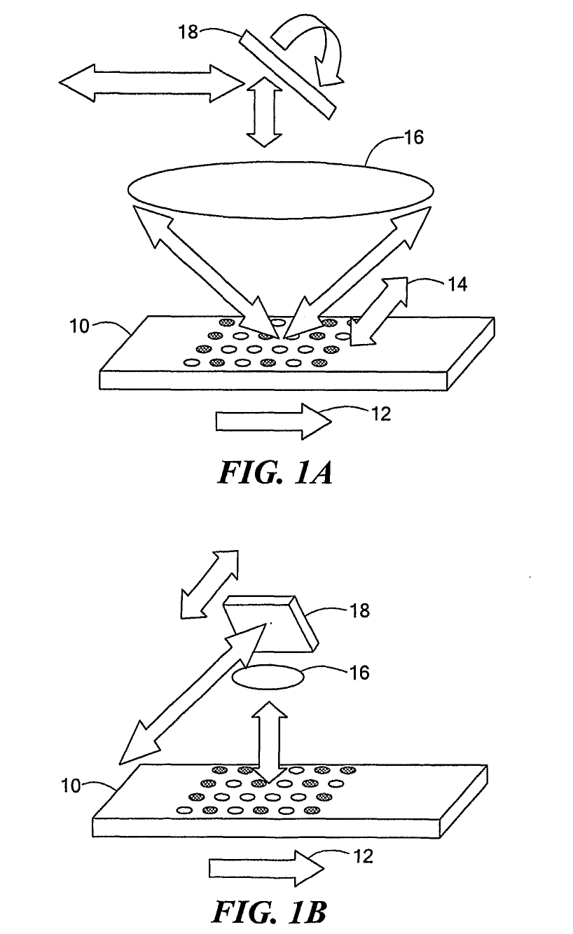

A FOI microslide of the invention can be used in each of the applications described below in reference to FIGS. 1 through 6. For example, a FOI microslide can be used in any application in substitute for or in combination with conventional substrates, microtiter plates, microarrays, microarray substrates, microscope slides, microarray plates or the like.

FIG. 1 shows three common scanning modes used in laser readers. (See Julian White, Genapta in Cambridge, England, United Kingdom., Pharmaceutical Discovery October 2004). Panel (a) illustrates a device that uses a fold mirror 18 mounted on a rotating galvanometer (not shown), where the deflected beam is focused onto the microarray 10 by a telecentric lens 16 that forms the objective. The telecentric lens allows the beam to be deflected at large angles by the mirror and still be focused onto a plane a few tens of microns thick at the surface of the array. One drawback is that telecentric lenses are relatively large, weighing hundreds of grams and costing approximately $2,000-$3,000 each. Panel (b) shows an alternative approach where both the fold mirror and the objective are moved back and forth over the smaller dimension of the array. Panel (c) defines a scenario in which the array is scanned in two dimensions.

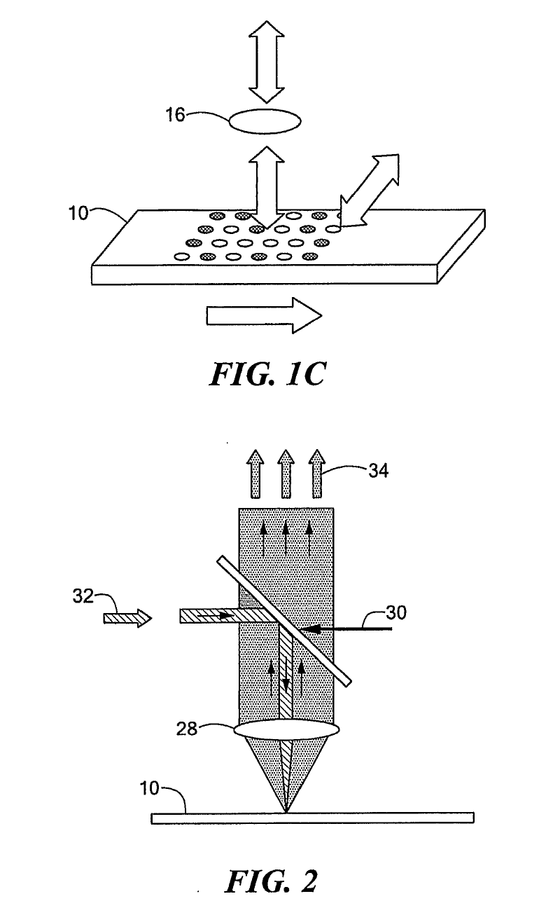

FIG. 2 shows a commonly used scheme for top down inspection of a microarray substrate 10. (See Performance Advantage of a Geometric Beamsplitter in ScanArray™ Microarray Scanners, Scan Array™ Technical Note 500, Packard BioChip Technologies, 40 Linnell Circle Billerica, Mass. 01821 USA, Web site: www.packardbioscience.com,array@packardbioscience.com). A laser beam 32 is used to excite a fluorescent reaction, which is detected through a focusing objective lens 28. A dichroic beam splitter 30 is used to separate the excitation beam from the fluorescent signal 34, which passes through to the detector.

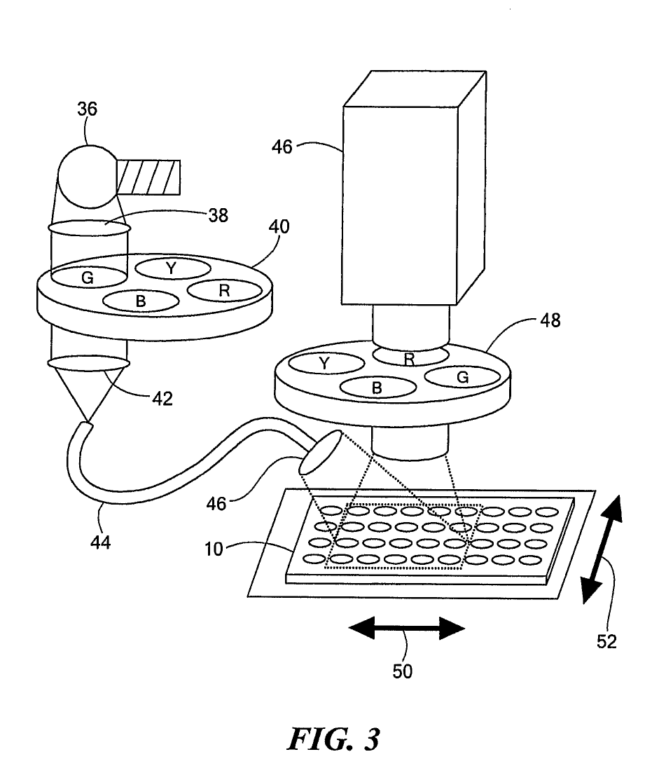

FIG. 3 shows the use of a CCD camera 46 for top down scanning of a microarray substrate 10. (See LaVision BioTec GmbH n Höfeweg 74 n D-33619 Bielefeld www.LaVisionBioTec.com). Fluorescent reactions are initiated by a light source 36 filtered 40 to isolate the excitation wavelengths 46. The CCD camera is also filtered 48 to exclude the excitation wavelengths, and only allow fluorescent emitted wavelengths to pass.

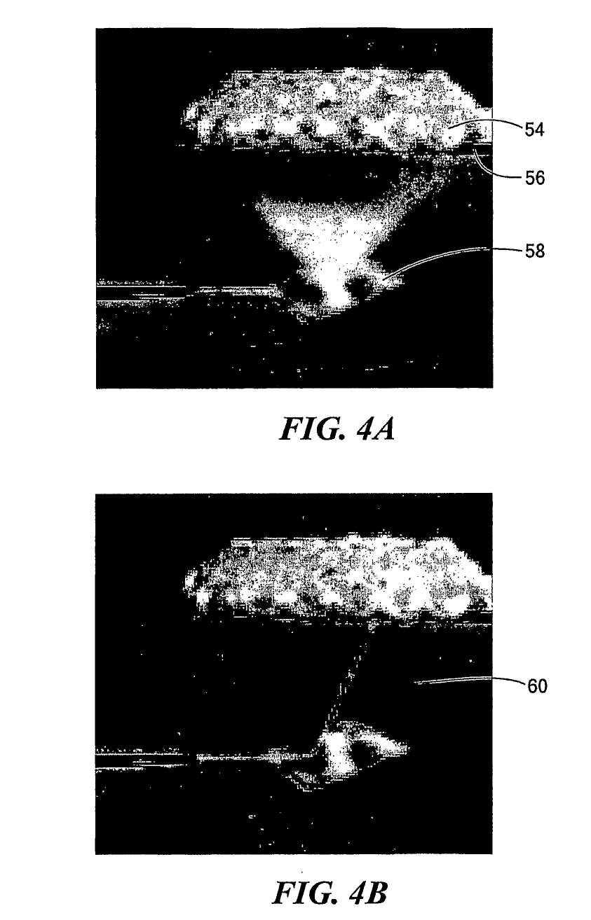

FIG. 4 shows an approach used for bottom scanning of microarray plates. (See Cyntellect, Inc. 6199 Cornerstone Court, Suite 111, San Diego, Calif. 92121-4740, web: www.Cyntellect.com). Fields of cells 54 are shown situated on the top of a very thin microscope slide 56. A high speed scanning galvonometer mirror 58 is used to illuminate the cells. In this scenario, the light 60 must pass through the thickness of the microscope slide substrate, and is subject to any distortion or optical effects that result.

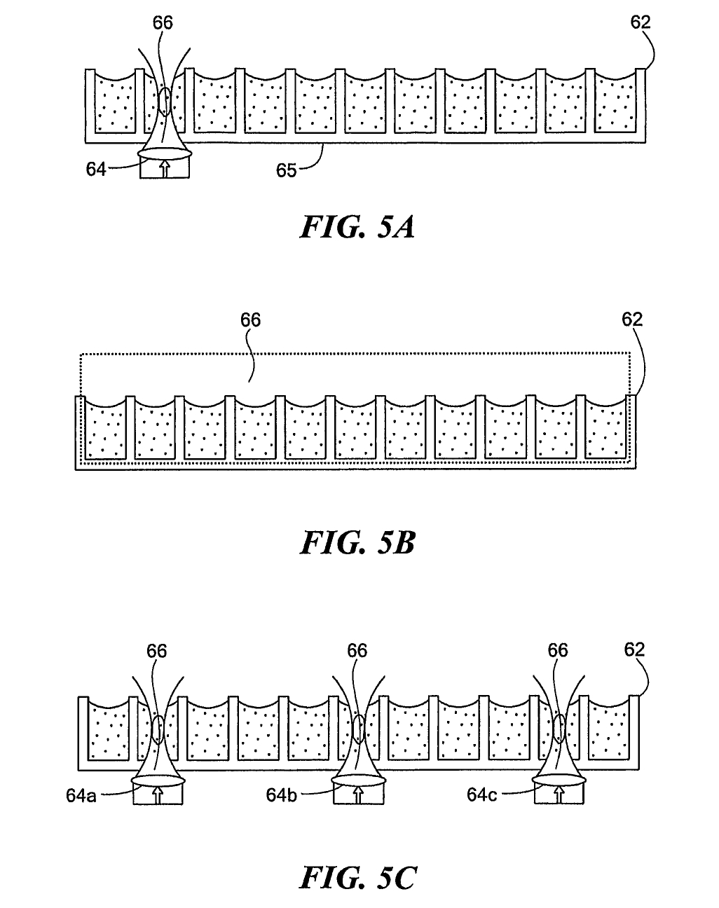

FIG. 5a depicts a microtiter plate 62 being optically interrogated by a single lens 64 that focuses light 66 through the plate bottom 65. (See Evotec Technologies GmbH, Schnackenburgallee 114, D-22525 Hamburg, Germany, www.evotec-technologies.com). Although individual wells are shown, the figure could as well be used to represent individual droplets, on the surface of a microarray substrate. This inspection scenario is limited by the slow rate of reading a multiwell plate or a microarray plate with many thousands of individual droplets. The middle pane (FIG. 5b) shows imaging of whole plate from above, which suffer from background and well to well (droplet to droplet) crosstalk issues that undermine the quality of the data. The bottom pane (FIG. 5c) shows a scenario in which multiple microlenses 64a-c are used to focus through the bottom thickness of the microtiter (or microarray) plate. This approach is a very expensive approach since each well or microarray spot requires a dedicated miniature lens aligned to interrogate each well or spot. Because of the finite size of these lenses, the approach is not easily-scalable to higher density microtiter (or microarray) plates.

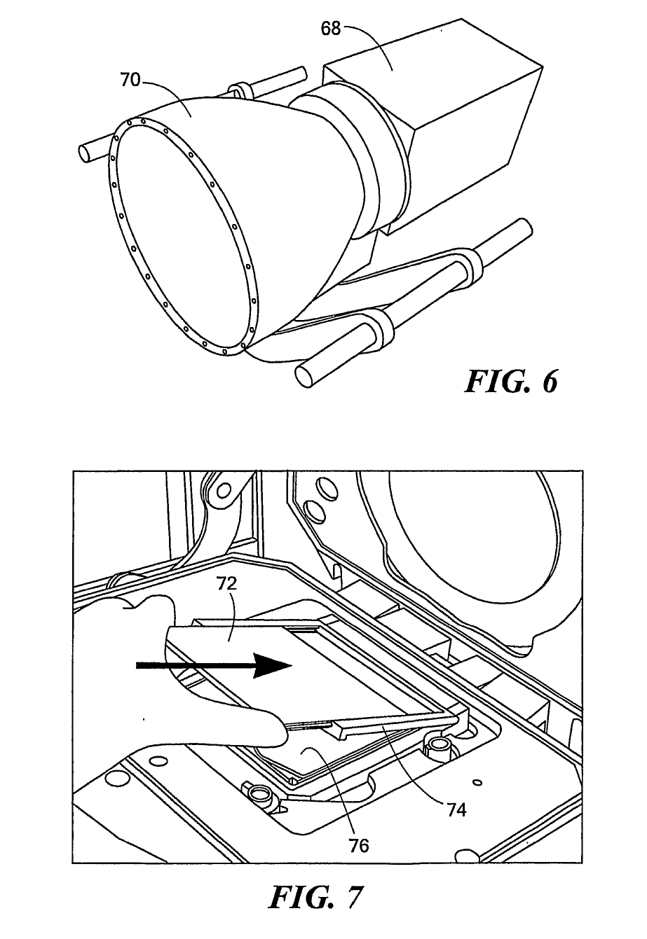

FIG. 6 shows a large format fiber-optic taper-coupled CCD camera. The light sensitive CCD chip sensor is housed in the white box 68. The black cone shaped portion 70 is a fiber optic taper which has been bonded directly to the CCD chip. The fiber optic taper is 200-mm in diameter and is an integral part of the camera.

Fiber Optic Interrogated Microslides according to the invention can be fabricated by bundling lengths of optical fiber and fusing them along their lengths. The fused bundle or block of fibers is then sliced into thin wafers such that opposing surfaces of each wafer consists of the proximal and distal ends of the optic fibers. FIG. 8a is a partial representation of a FOI microslide 72 integrally composed of optic fibers 78 comprising a central core glass region surrounded by clad glass. FIG. 8b represents an embodiment having two FOI microslides 72 stacked one upon the other. Preferably, the optic fibers of each microslide are aligned to be in register with the optic fibers of the other. A surface of the first microslide substrate is adjacent to and contacting a surface of the second microslide substrate. Given that each microslide is capable of zero thickness optical equivalence, the microslides of the invention can be stacked, preferably with their surfaces in direct physical contact, and then bound together once the optical pathways are aligned. Stacking of microslides can be useful in optically coupling, for example, a sample to an imaging device or sensor array.

The plurality of optic fibers are shown to be bundled and fused together by the clad glass. The fibers may be bundled and fused together by any suitable technique such as those used in standard manufacturing practices. The plurality of optic fibers may also be coupled to conventional detection equipment including at least one charged coupled device (CCD).

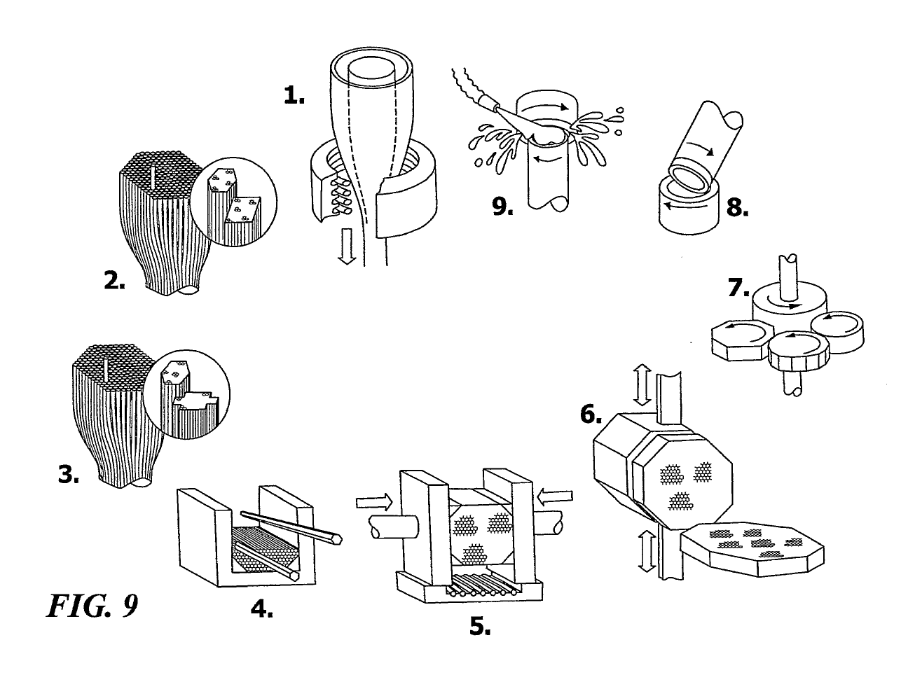

FIG. 9 is a pictorial representation of the manufacturing process used to produce Fiber Optic Interrogated Microslides. The starting point is a core glass rod, sized to fit closely within a clad glass tube. Together they are loaded into a furnace where they are fused and drawn into long lengths of cane, typically about 2.5 mm in diameter. Long lengths of cane are assembled into billets, which are re-drawn forming the first ‘multi’. The process is repeated, with ‘multi’ assembled into a second billet, which is drawn again to form ‘multi-multi’ cane. During the “mold load” stage, “multi-multi's” are cut to the desired block length and stacked into a pressing fixture (typically about the size of a loaf of bread). The assembled mold is placed into a pressing furnace. During ‘pressing’, the furnace heats and softens the fiber array, while a load is applied. The block is then annealed and fabricated into finished product. For a fiber optic array plate of the invention, block material is cut into rectangular plates having the desired nominal thickness. Plates are ground and polished to target dimensions using glass finishing slurry and pad materials.

The process described by FIG. 9 is not limited to glass materials. Optical fibers manufactured using plastic materials can be formed into Fiber Optic Interrogated Microslides, using processing techniques similar to that described in FIG. 9. The final plate comprises fiber optics that effectively transfer optical images from one surface of the microslide to the other. The finished faceplate of the invention is the optical equivalent of a zero thickness window that can also be used for field-flattening, distortion correction and contrast enhancement.

### Optical Characteristics Of A Fiber Optic Interrogated Microslide Of The Invention

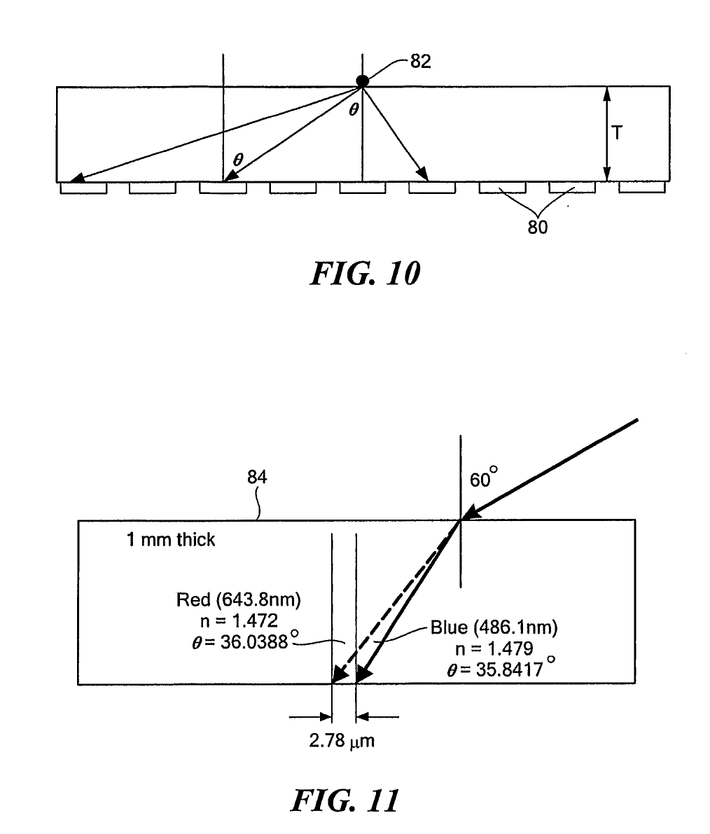

The purpose of this section is to describe the optical performance of a Fiber Optic Interrogated Microslide and to show how that performance is differentiated from a conventional (glass or plastic) substrates, microtiter plates, microarrays, microarray substrates, microscope slide, microarray plates or the like. First, the optical performance of a conventional microscope slide is considered in particular, how an object or a light source on the surface of a conventional microscope slide would be imaged onto a sensor or detector, in contact with the bottom of the microscope slide. FIG. 10 shows a conventional microscope slide, having a thickness ‘T’. A sensor or detector 80 in contact with the bottom of the slide is depicted (rectangles) below the slide. This sensor or detector could be a CCD array, or photosensitive film or other appropriate material and, alternatively, used with an embodiment of the invention. If a light source 82 (shown as a dot) on the surface of the microscope slide radiates in all directions (360 degrees), half of the light radiates in the opposite direction of the slide, and is lost. The other half (θ=180 degrees) radiates in the direction of the slide and is either transmitted or reflected back at the interface depending on the refractive index of the glass and surrounding medium. The light entering the slide transmits through the glass and propagates at all angles (θ) as shown in the figure. Depending on the thickness of the slide (typically 1-2 mm), the light will spread as it propagates through to the other side. If two adjacent light sources are considered, the ability to resolve the two will depend on the extent to which the light spreading from each overlaps the other.

The extent of this spreading can be calculated using equations available in reference 1. The result of this analysis, as well as practical experience, demonstrates that the light spreading from closely spaced sources overlaps, destroying resolution. A microscope slide is an ineffective means of imaging sources onto a CCD array without intervening optics. If a sophisticated, properly designed lens system is available to focus through the glass slide, an image of the light source can be reconstituted. Commercial systems equipped with sophisticated optics (inverted microscopes for example) are available; however they are very expensive. In addition, conventional optics causes other distortions such as chromatic aberration. FIG. 11 shows the effect of chromatic dispersion. The refractive index of many glasses varies depending on the wavelength of light. Differences in the refraction for different wavelengths causes distorted images, sometimes seen as a halo effect. In a borosilicate glass microscope slide 84, the resulting displacement between the red and blue beams is typically several microns. For the example shown in FIG. 4, the displacement is 2.78 μu. For a high-resolution lens system, this effect would be detrimental, resulting in a red-colored halo around the outside of the image and loss of resolution.

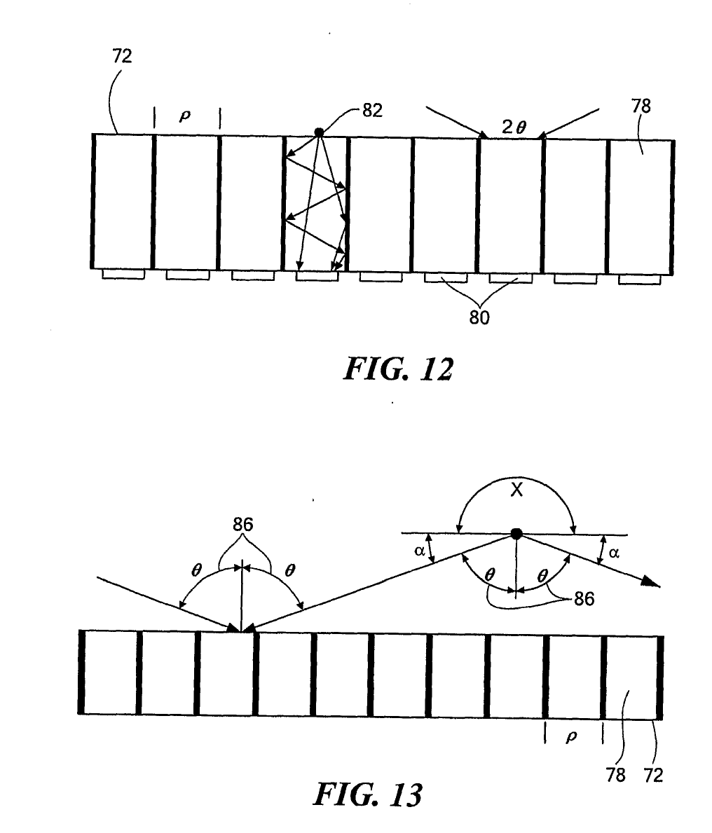

FIG. 12 depicts the optical performance of a Fiber Optic Interrogated Microslide 72 of the invention and shows how it is differentiated from a conventional microscope slide. A Fiber Optic Interrogated Microslide is comprised of individual optical fibers that conduct light incident on one face to the opposing face. Each of the constituent fibers comprises a high-index glass core surrounded by a lower-index optical cladding, so that the resulting multimode fiber guides the light. The figure illustrates a parallel array of individual fibers 78, with the higher refractive index core glass, separated by the surrounding clad glass (black lines). A light source 82 (shown as a dot) is shown on the surface of the slide, contacting the proximal end of an individual fiber optic core. A sensor or detector 80 in contact with the bottom of the slide is depicted (rectangles) below the slide, in direct contact with the distal end of an individual fiber optic core. This sensor or detector could be a CCD array, or photosensitive film or other appropriate material.

The optical properties of the Fiber Optic Interrogated Microslide can be dependent on fiber dimensions (core and clad dimensions) as well as the refractive index properties of the respective materials. Together, these parameters determine the numerical aperture (light gathering) and the modal properties (light guiding) of the microslide.

Each of the constituent fibers consists of a high-index glass core surrounded by a lower-index optical cladding, so that the resulting multimode fiber guides the light. Using the ray approximation for meridional waves, the acceptance angle θ of the fiber is given by:

n₃ sin(θ)=\sqrt{n_{core}²−n_{cladding}²} Equation 1

where θ is the acceptance angle of the microslide, n_{core} is the refractive index of the fiber core, n_{cladding} is the refractive index of the cladding, and n₃ is the refractive index of the material immersing the input end of the fiber. For air, n₃=1. The numerical aperture (NA) of a fiber is defined to be:

NA=\sqrt{n_{core}²−n_{cladding}²} Equation 2

In some instances, the left side of Equation 1 is defined as the numerical aperture, which leads to ambiguity in interpretation if the radical is greater than unity. In the present context, Equation 2 is used since it is an unambiguous number associated with the material properties of the fiber. This form also enables easy extrapolation to various external refractive indices. Values for NA are shown in Table 1 for some common glass materials (partial list only) used in microslides. For NA>1 and n₃=1 (air), Equation 1 has no mathematical real solution for sin(θ)>1, so the maximum possible acceptance angle of 90° is listed for light approaching from outside the fiber. When the calculated NA>1, it merely means that light propagating inside the fiber could bounce (be guided) at larger angles than is possible to excite by shining light from outside the fiber. X26, X15, C5, M1 and C1S refer to different glass composition that could be used to fabricate m croslides of the invention.

TABLE 1
Typical NA's calculated based on core and clad refractive index.

| Core | | Clad | | | | Acceptance | Core |
|---|---|---|---|---|---|---|---|
| Composition | | Composition | | Specified | Calculated | Angle (θ) | Diameter |
| (Index) | | (Index) | | NA | NA | (in air) | ρ |
| X26 | (1.87) | C5 | (1.49) | 1.0 | 1.130 | 90° | 9, 6, 12μ |
| X14 | (1.80) | C5 | (1.49) | 1.0 | 1.010 | 90° | 3-10μ |
| X26 | (1.87) | C1S | (1.48) | 1.0 | 1.143 | 90° | 3-10μ |
| M1 | (1.625) | C1S | (1.48) | 0.66 | 0.671 | 42.1° | 6-8μ |

Light Detection with Microslides of the Invention

FIG. 13 illustrates the capture of radiation from an isotropic point source for a fiber with NA<1 (acceptance angle less than 90°). Only light radiated downward into the cone with vertex angle 2θ (86 in FIG. 13) is captured in guided modes of the microslide 72. (A small percentage of the light is also reflected at the interface between air and glass.)

Light radiated into the fan of angle α strikes the microslide, but exceeds the NA of the fiber and is either absorbed by the fiber cladding and EMA glass within the microslide or reflected from the surface. EMA glass is “Extra-Mural Absorption” glass. It is a highly absorbing glass that is incorporated into a faceplate in one of several schemes. The EMA glass isolates each fiber optically so no cross talk occurs when light enters the fiber at angles that exceed the NA of the fiber. Such light is not guided by the fiber, but instead, propagates into the cladding. Without the EMA glass, this light has the potential to scatter into propagating modes of an adjacent fiber, causing cross talk. The EMA glass absorbs any light that penetrates into the cladding before it can reach adjacent fibers. Light radiated into the hemisphere denoted by X is radiated away and never reaches the microslide.

If the NA of the microslide is increased, the angle θ increases and a greater percentage of the light is captured. However, as θ increases, the light spreads across more fibers of the microslide of the invention, degrading the resolution, unless the source is directly in contact with the microslide. (In this context, resolution refers to the ability to measure the lateral location of the light source by viewing it through the microslide. As the light from the source spreads to more and more fibers, the image is less localized, so the resolution is poorer.) If the point source is in contact with a fiber in the microslide, all of the downward-radiated light is captured by that fiber.

There are additional considerations when a microslide includes a taper. A taper in a microslide acts as a magnifier or reducer. This property can also be incorporated into FOI microslides.

All of the light emitted downward within the NA of the fiber is captured entirely by that fiber and is conducted from the proximal to the distal end of the fiber where it is detected (photosensitive film, CCD array etc.). The benefit of this construction is that the light is transmitted so that it can be detected without requiring any additional intervening focusing optics. For example, a CCD array can be used for direct contact imaging if the CCD pixel is properly sized with respect to the fiber diameter ρ. This capability represents one of the strongest attributes of microslides.

The image resolution achieved with the FOI Microslide of the invention can depend in part on the dimensions of the individual fibers making up the slide, the resolution of the detector, as well as the size of the object being viewed relative to the fiber and detector dimensions. In the present context, resolution is interpreted as the smallest element or separation that can be resolved when viewing objects through a microslide. Therefore, a smaller or finer resolution is better. Sometimes confusion can result because a system may be referred to as “high” resolution, which actually implies the ability to resolve smaller elements. The diameter of the fiber (ρ as shown in the figure) can be controlled depending on the details of the manufacturing procedure, and can range from less than 3 microns to over 2,000 microns. The pixel size of CCD detectors varies from 6-30 microns, but is commonly about 9-10 microns. Photographic film has a distribution of grains sizes; however the average is found to be between 0.8-3 microns. For most FOI microslide applications of the invention, the fiber can be either 3 microns or 6 microns in diameter, well matched to the size magnitude of the film or CCD detector. For many biological applications, the size of the object being viewed is also well matched to the resolution limits imposed by fiber size and sensor pixel or grain size. For example common mammalian cell diameters range from >2 to <10 microns in diameter. For many biological applications of a microslide of the invention, the purpose may not be to image an object, but to detect light that is emitted as a result of a fluorescent or luminescent reaction.

The resolution of a microslide of the invention is excellent for many applications since it conveniently matches the resolution available in common CCD devices. For example, FIG. 12 depicts a light source (object being viewed) that is considerably smaller than the diameter of the interrogating fiber. In that case, the light emitted by the light source would fill the fiber as shown by the arrows, and the ‘image’ of the light source would be the same size as the fiber itself. Since the fiber diameter can be 3 microns or less, and typical CCD pixel size is 9-10 microns, the microslide preserves and does not detract from overall system resolution which in this case would be governed by the pixel size of the CCD.

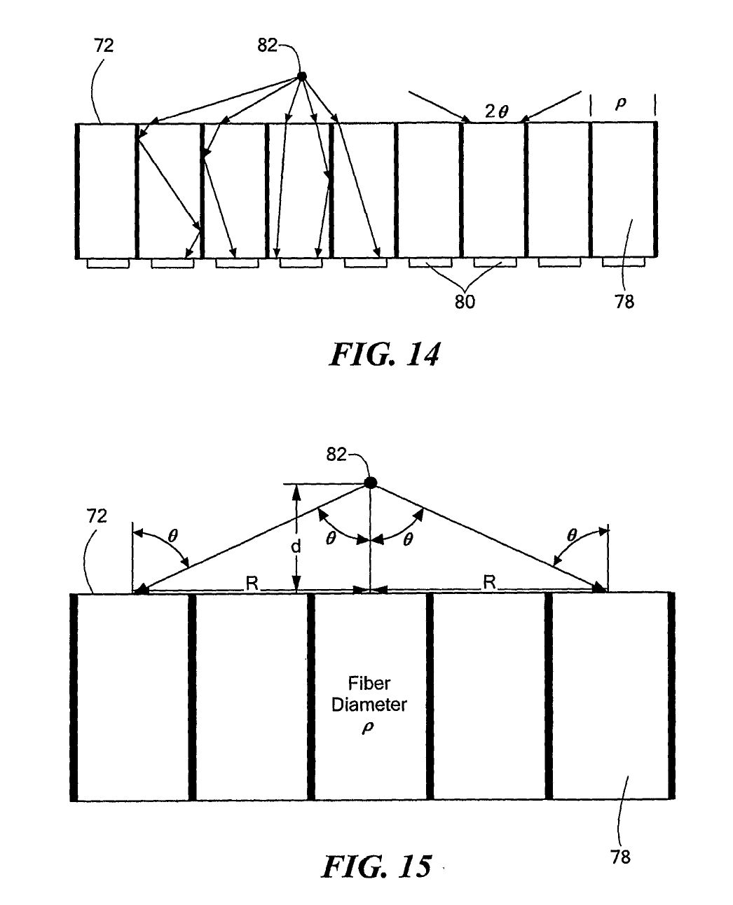

FIG. 14 depicts a different scenario in which the light source 82 (object being detected) is suspended in a medium (air, liquid, etc.) above the surface of the microslide 72. The emitted light might originate as a result of a fluorescent or luminescent reaction, used as an indicator to in an analytical or diagnostic technique. A liquid droplet containing samples of diagnostic interest might be freely sitting on the surface of the microslide, restrained by its own surface tension, as might be the case for a droplet spotted onto the surface using ink jet printing, split pens, or other techniques, to form a microarray. Alternatively, the surface of the microslide might be patterned to form wells that retain a liquid droplet containing samples of diagnostic interest as in the case of a microwell array. When exposed to appropriate reactants, the samples of diagnostic interest might emit a light signal. In either of the exemplary scenarios described (droplet or microwell), the light emitted from the light source would distribute itself across multiple fibers 78, with varying intensity depending on their radial distance from the source, and the numerical aperture (acceptance angle θ, see FIG. 14) of the fiber. This light signal could then be detected by a sensor or detector 80 in contact with the bottom of the microslide. Properly detected, this light signal would provide a definitive indication of the status of the reaction occurring within the droplet or well of interest. It is assumed that the light source is small compared to the size of the droplet or well and that the droplet or well is interrogated by 1 or more fibers, although such may not always be the case.

Imaging with Microslides of the Invention

Certain other applications of the invention can include imaging rather than detecting the light source. As previously discussed, if the light source object is in direct contact with the surface of the microslide, the object can be imaged without loss of resolution, provided that the object is approximately the same size, or larger than the fiber size. If the light source object is located above the surface of the microslide, the object can also be imaged. For many applications, direct interrogation through a microslide offers significant cost advantages compared to more complex focusing optics.

The effect of ‘distance above the microslide’ can be analyzed to determine the impact on imaging resolution. For a light source located above the microslide a distance d, there will be a circle of illumination of radius R on the microslide surface over which the light arrives within the acceptance angle θ of the constituent fibers. As shown in FIG. 15, the radius R of this acceptance surface on the microslide is:

R=d tan(θ) Equation 3

The diameter, 2R, of the acceptance circle represents the minimum resolution possible when looking at the source through the microslide of the invention. In practice, the resolution can degrade in increments of fiber diameter p because the output side of the fiber is either illuminated across its entire aperture or is not illuminated at all. The acceptance angle as seen from the source 82 is equal to the acceptance angle at the fiber face by the well-known Euclidean geometry theorem that angles on opposite sides of a transversal between two parallel lines are equal.

As is evident from the drawing, if R<ρ/2, then only the central fiber is illuminated. (This approximation ignores the subtleties of whether the source is centered on the fiber or is located close to an edge.) This can place a limit on the height of the source d above the surface to avoid degradation of the resolution beyond the intrinsic resolution ρ of the microslide:

R=d tan(θ)≦ρ/2 [symbol] d≦ρ/2 tan(θ) Equation 4

For every additional increase in R by a length ρ, a new ring of fibers is illuminated, which can degrade the resolution and spread the light from the single point source across more pixels of the CCD array. If a pixel is partially illuminated, the brightness of the fiber as detected by the CCD can be reduced proportionally, although the fiber may be uniformly illuminated across its cross section because the bound modes spread throughout the fiber. Also, if the brightness of the source varies with emission angle θ, the brightness received by the CCD array can be further modified. The specific radiation profile must be integrated to calculate the resolution if the source is located above the microslide. However, in general, if the radiation profile is contained with an angle θ from the normal, it can have the same effect as a microslide with an NA of the same value.

In Table 2 the maximum elevation of a source that avoids significant degradation of the resolution is shown for several microslide parameters. The effective NA is unity and θ6=90° so tan (θ)≈∞ for some combinations of core and clad glass. For these microslides of the invention, (NA=1) the point source should be in contact with the surface to maintain the resolution according to Equation 4. Nonetheless, the variation of the received power with angle reduces the severity of the contact requirement slightly.

TABLE 2
Maximum source height for best resolution with

| Fiber | Numerical | Acceptance | Maximum |
|---|---|---|---|
| Diameter | Aperture | Angle (θ) | source height |
| (ρ) | (NA) | (in air) | (d) (Eqn. 5) |
| 3 μm | 0.671 | 42.1° | 1.7 μm |
| 3 μm | 1.010 | 90° | 0 μm |
| 6 μm | 0.671 | 42.1° | 3.3 μm |
| 6 μm | 1.143 | 90° | 0 μm |

### Exemplary Microslide Designs

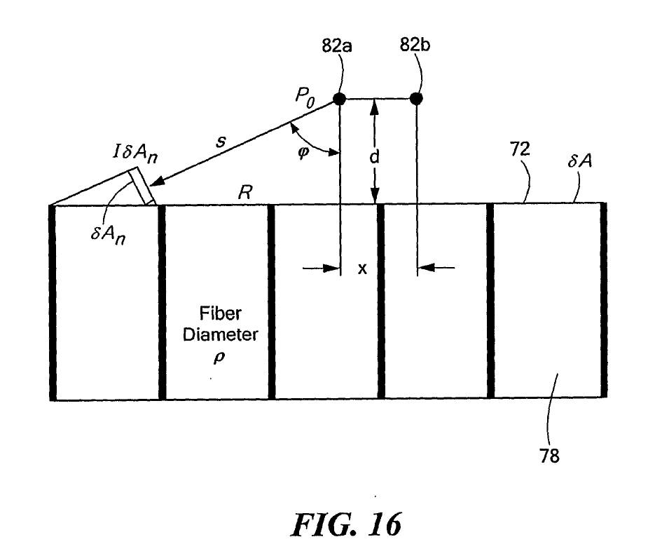

FIG. 16 depicts an isotropic point source 82a of total optical power Pₒ located a height d above the microslide surface. A second source 82b separated a lateral distance x from the first one is also shown for later reference. The power received by an arbitrary fiber from a single source is:

P_{f}=I(s)δAₙ=PₒδAₙ/4πs² Equation 5

where P_{f} is the optical power received by the fiber, Pₒ is the total optical power radiated by the isotropic source, s is the distance from source to the fiber, I(s) is the optical intensity at the source, Φ is the angular direction of the fiber relative to the normal to the source, and δAn is the projection of the cross sectional area of the fiber normal to vector s:

δAₙ=πρ² cos(φ)/4 Equation 6

Since d=s cos(Φ), Equations 3 and 4 reduce to:

$${P}_{f} ( ϕ ) = {P}_{o} {ρ}^{2} {cos}^{3} ( ϕ ) / 16 {d}^{2} = \frac{{P}_{o} {ρ}^{2} d}{16 {( {R}^{2} + {d}^{2} )}^{3 / 2}} \qquad Equation 7$$

This formula only holds for d≧ρ because for lesser values of d, the angle ρ changes significantly across the fiber aperture ρ and must be properly integrated to give correct absolute power. Defining the normalized received power P_{N} as the power received by a fiber at angle Φ, it follows that:

P_{N}(φ)=P_{f}(φ)/P_{f}(0)=cos³(φ). Equation 8

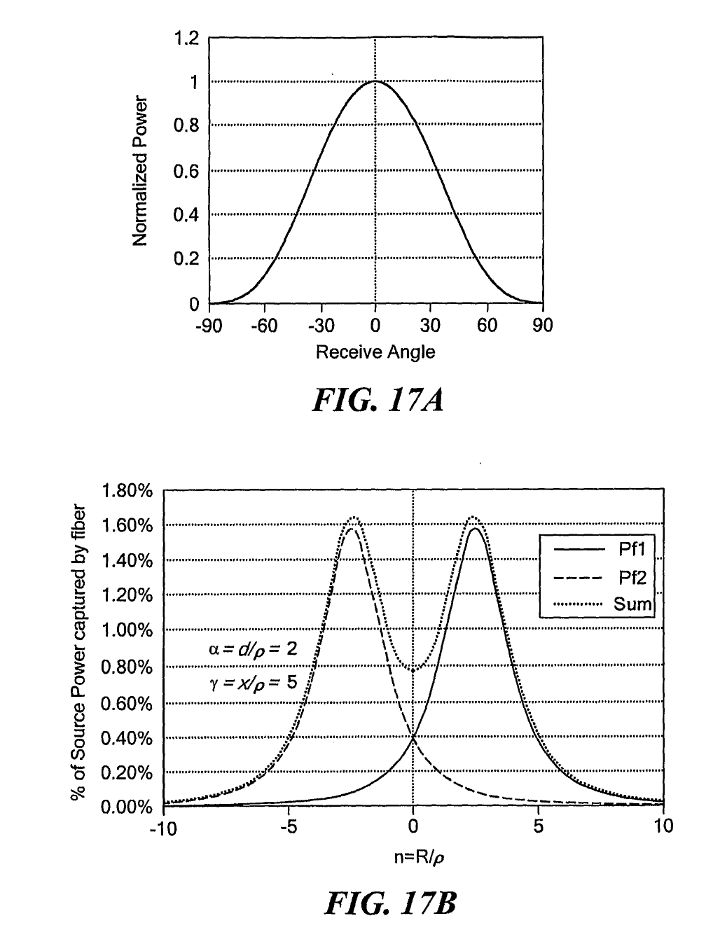

This relationship is graphed in FIG. 17 (left side). The graph shows that even for an isotropic source above a microslide of the invention with NA=1, the received optical power per fiber can have a maximum directly beneath the source that decreases as the angle of the fiber from the source increases as shown in equation 8.

For two sources separated laterally by a distant “x” as shown in FIG. 16, the analysis from Equation 7 can be extended to:

$${P}_{Tot} = \frac{{P}_{o} {ρ}^{2} d}{16} ( \begin{matrix}\frac{1}{{( {( R - x / 2 )}^{2} + {d}^{2} )}^{3 / 2}} + \\ \frac{1}{{( {( R + x / 2 )}^{2} + {d}^{2} )}^{3 / 2}}\end{matrix} ) \qquad Equation 9$$

This falloff can be shown by the falloff in FIG. 17 (right side) in which small heights of the source above the microslide surface are expressed in multiples of the fiber diameter ρ where P_{f1}, and P_{f2} are the powers captured by the fiber from sources 1 and 2. The percentage of light captured by each fiber declines rapidly with height. The intensity profile does allow two discrete sources to be resolved under certain conditions. A numerical evaluation of Equation 9 is shown in FIG. 17 (left side) for the case that the source elevation is twice the fiber core diameter and the source separation is 5 times the fiber core diameter. It can be shown that two distinct peaks are observed (resolved) if the source separation is about 2.5 times greater than the elevation above the microslide of the invention.

In general, for point sources located above the top surface of the microslide of the invention without any intervening fill fluid, the square-law decline of optical power and the projection of the fiber cross-sectional area can allow point sources to be laterally resolved. From the image side, the point sources can appear as a double-humped light distribution. This condition for the double-humped distribution can be inferred by the following: Two point sources separated by a distance ‘S’ may be resolved, provided that the source separation S>2.5 d where d=source height.

### Uses Of FOI Microslides

FOI microslides are one contribution to this growing market that can be used in conjunction with such high speed analytical techniques. The market for a FOI microslide includes pharmaceutical, biotechnology, and agricultural companies as well as universities and research institutions. Applications include: drug discovery, life science research, in vitro diagnostics, disease management, forensic medicine, and drugs-abuse testing.

FOI microslides represent a radical departure from the traditional design of plain microscope slides. Plain microscope slides are the familiar clear rectangular homogeneous glass plates used to hold specimens for examination under a microscope and cover glasses are the smaller, thinner glass plates used on the microscope slides to cover specimens for protection during examination. Plain microscope slides and cover slips are examples of plate glass substrates that can be replaced in many applications with FOI microslides of the present invention. FOI microslides improve the accuracy and resolution of automated inverted microscopy, and allow direct CCD contact imaging. The effects of distortion associated with conventional glass slides are eliminated providing a series of novel features and advantages not realized with conventional microscope slides. FOI microslides could replace conventional microscope slides in a number of applications, including use as a microscope slide for inverted microscopy, as a substrate for a microarray, and in glass bottomed microtiter plates. The FOI microslide can be marketed 1) as a stand alone product, 2) combined with specialty coatings that enable certain applications for biological and other applications and 3) as part of a kit designed to enable biotechnology and other applications. Many other applications are expected to evolve to take advantage of this substrate material.

### Application Example of a Microarray Microslide of the Invention

Various techniques are used to ‘print’ or ‘spot’ samples onto a microarray substrate of the invention. Ink jet printing and split pin printing are in common use. These techniques are used to deposit a droplet of sample on the surface of the substrate. Various substrate coatings (described later) can be used to insure good bonding of the biological sample to the substrate. Other coatings can make the surface of the substrate hydrophobic, and insure that closely spaced droplets remain separated and do not flow or diffuse into each other.

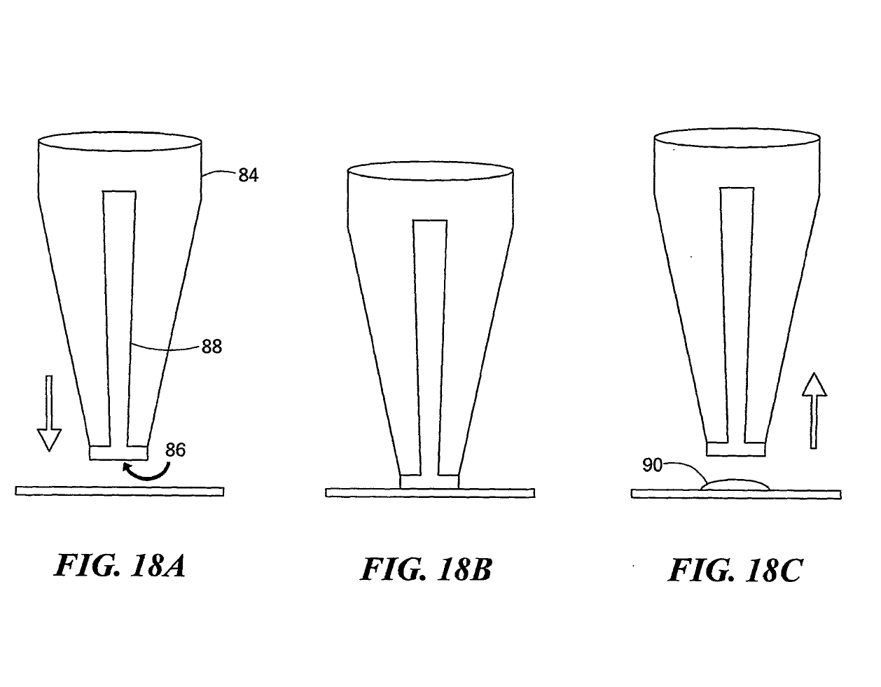

Split pin printing enables high-speed manufacture of microarrays on microslides and other microarray surfaces of the invention. Split pins work on the same principal as vintage dip pen tips used for writing. As shown in FIG. 18, the split pins 84 have flat tips 86 and defined uptake channels 88, which allows a thin (25 μm) layer of liquid sample to form at the end of the pin, and printing to proceed by gentle surface contact. As shown in FIG. 18 printing occurs as a simple 3-step “ink-stamping” process as follows: (a) down stroke, (b) contact, and (c) upstroke. Pin tips and channels are available in a wide assortment of dimensions, allowing users to specify spot diameter and the number of spots per loading.

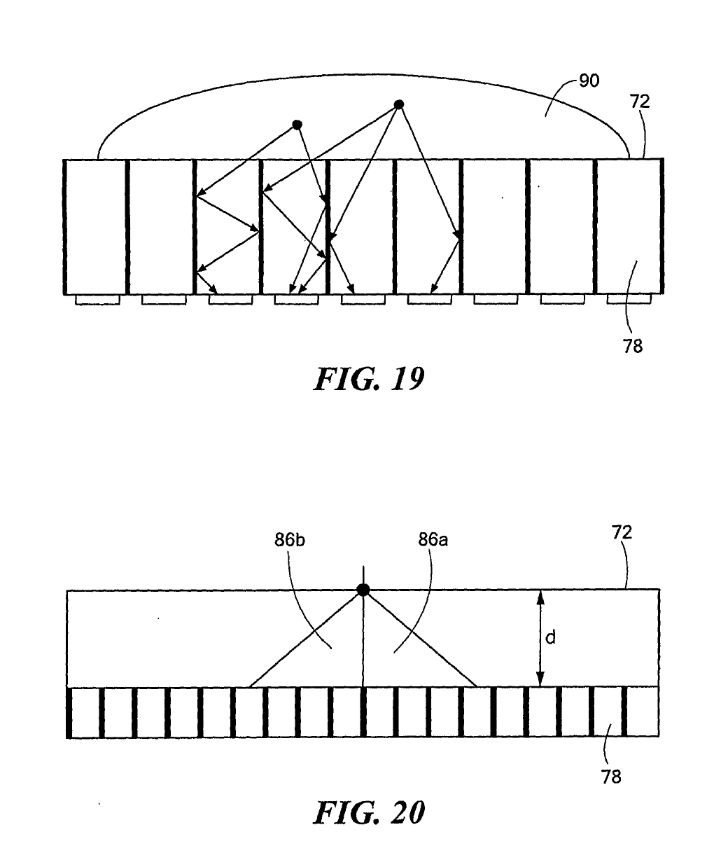

A microarray droplet 90 is formed on the top of a FOI microslide 72 as depicted in FIG. 19. To estimate the resolution within a droplet, the refractive index of the fill fluid should be considered. For biological studies, the droplet is presumed to be filled with a fluid with refractive index near that of water, 1.33. For a faceplate with a NA=1 in air, the acceptance angle may be decreased according to Equation 1.

If a fluorescent source may range throughout the depth of the spot, then the ability to resolve the lateral location of the fluorescent source within the spot will vary with the height. If the source moves in contact with the surface of the microslide, the lateral resolution can be approximately equal to the fiber diameter. However, if the source moves near the top of the droplet, the resolution may degrade.

The droplet is applied by standard microarray spotting techniques, and is large enough to conduct biological studies therein. The “fluorescent” reactions within the spot can be optically monitored via one or many microslide fibers interrogating each droplet. Exemplary calculated diameters (2R) on the surface of the microslide that accepts light from a point source in the droplet are shown in Table 3. Results from Equation 4 are shown for various source heights and in air and for water-filled droplets. The radius R of the acceptance circle is the same as shown schematically in FIG. 15. As discussed previously, the microslide can transmit any light incident within the cone defined by the acceptance angle θ. As the height d of the source increases, the radius R illuminated by light within the acceptance angle (assuming θ<90°) increases. As more fibers are illuminated within the acceptance cone, the resolution of the system may degrade.

TABLE 3
Diameter of circle of acceptance with microslides of the invention.

| Numerical | Acceptance | Acceptance | | | | | |
|---|---|---|---|---|---|---|---|
| Aperture | Angle (θ) | Angle (θ) | | | 2R | 2R | |
| (NA) | (in air) | (in water) | d | | (in air) | (in water) | |
| 0.671 | 42.1° | 30.3 | 5 | μm | 9.0 μm | 5.8 | μm |
| 0.671 | 42.1° | 30.3 | 200 | μm | 362 μm | 234 | μm |
| 1.010 | 90° | 49.4° | 5 | μm | ∞ | 11.7 | μm |
| 1.010 | 90° | 49.4° | 50 | μm | 4 | 117 | μm |
| 1.010 | 90° | 49.4° | 200 | μm | 4 | 467 | μm |
| 1.143 | 90° | 59.3 | 5 | μm | 4 | 16.8 | μm |

If a fill fluid of index n₃ is added, Equation 1 indicates that the acceptance angle decreases. In this case, the radius R or the acceptance cone is decreased and fewer fibers may be able to guide the incident light. Light incident at angles greater than the acceptance angle is absorbed rather than guided by the fibers. For a fluid droplet, a smaller fraction of the source energy may be emitted into the smaller acceptance angle in the fill fluid. The light may be guided by fewer fibers so the resolution of the interrogation system improves. Although much more of the emitted light may be rejected in the presence of a fluid droplet, the brightness of the image viewed through the central fibers is the same. This effect is illustrated in FIG. 20 for a microslide with a calculated NA=1.010. In air, generally all of the light emitted downward at angles of 90° or less (gray and yellow areas) is transmitted by the recipient fibers. When a fluid droplet with index of 1.33 is added, the acceptance angle can fall to 49°. All of the light emitted into the gray area may no longer captured by the outlying fibers, but all of the light emitted into the yellow can continue to be captured and transmitted. Therefore, the addition of the fluid merely prevents the outlying fibers from transmitting light and degrading the system resolution.

For a droplet on a microslide, that is large compared to the dimension of the fiber, a luminous source located near the top of the droplet would illuminate the entire droplet bottom within the acceptance angle. Generally, in this case, no information is obtained regarding the lateral position of the source within the droplet.

If the source is close to or in contact with the bottom of the droplet or preferably on the microslide surface, and the droplet diameters is 2 to 10 times larger than the diameter of the acceptance circle on the microslide surface, useful information about the location or motion of a luminescent source within the droplet, could be determined. This ability may be useful, for example in studies of cell migration within the fluid.

### Direct Contact Viewing of Microslide Microarrays of the Invention

Direct contact viewing of microslides microarrays of the invention is generally analogous to contact printing in photography. In photographic contact printing, the negative is placed in direct contact to the photographic paper and exposed. Images on the negative are captured on the photographic paper, without the requirement of complex focusing lens systems (such as are used with an enlarger). Furthermore, the entire image on the negative is captured simultaneously, without having to scan. Direct contact viewing of microarrays formed onto conventional microscope slides is not possible since, as previously described; the loss of resolution, for light traveling through the thickness of the slide is unacceptable. Fiber Optic microslides behave optically like a zero thickness substrate. Light signals or images on the top surface of the microslide transmit to the bottom surface with a fixed resolution that depends on the fiber diameter. Direct contact viewing can be used to image the microarray using the following scenarios:
a) Direct contact of the microslide microarray to a light sensitive medium such as photographic film (analogous to photographic contact printing)
b) Direct contact of the microslide microarray to a light sensitive electronic sensor such as a charge coupled device (CCD). Since CCD chips are typically small, and environmentally sensitive, an arrangement may involve a CCD camera incorporating a CCD chip bonded to a protective fiber optic microslide or taper.

FIGS. 1-5 depict various ‘indirect’ strategies that can be used for viewing of conventional microarrays and/or an embodiment of the invention. These techniques are characterized as indirect since in all cases the microarray is imaged onto a sensor or detector such as a CCD or photomultiplier tube, using various combinations of mirrors and lens that can be scanned or focused to direct the light to the detector. Bottom viewing through the conventional glass slides used for some microarrays is complicated by the distortions, and loss of resolution that results from viewing through a thickness of glass. As a result, complex mechanisms must be employed to gather light from the microarray and attribute it to the appropriate closely spaced spot. Some of the scenarios depicted also include optical filtering strategies to separate fluorescence excitation and emission wavelengths. As indicated above, these same ‘indirect viewing’ techniques can also be used to view fiber optical microslides of the invention. In most cases however, the mechanisms can generally be simplified since they may be focused on the bottom surface of the microslide.

FIG. 6 shows a CCD camera which incorporates a fiber optic microslide/taper as an alternative to a lens system. The light sensitive sensor is housed in the white box. The black cone shaped portion is a fiber optic taper. The fiber optic microslide/taper provides environmental protection to the sensitive CCD chip, and guides light to its surface. Fiber optic microslides of the invention can be bonded directly onto the CCD or CMOS imagers to provide vastly improved image resolution compared to lenses. Large format CCD cameras can incorporate a fiber taper which allows images to be gathered over a large area to be detected by a much smaller CCD chip. Fiber bundles incorporating extramural absorption (EMA) fibers minimize optical crosstalk between fibers and improve contrast. Fiber bundles can range in magnification from 1:1 fiber microslides to large 6:1 fiber tapers, and in diameters up to 200 mm. The fiber optic taper shown in FIG. 6 is 200-mm in diameter. It is an integral part of the camera, and is bonded directly to the CCD chip.

FIG. 7 depicts the direct contact viewing of the microarray, which is enabled by using a fiber optical microslide substrate 72 of the invention in direct contact with the faceplate of the CCD camera. The faceplate of the CCD camera is made from the same fiber optic construction that is used to produce the microslide. The faceplate of the CCD camera can be a permanent part of the camera. The microslide can be a removable, interchangeable, and in some applications a disposable ‘sample carrier’. Furthermore, the microslide may have specially applied coatings on one surface to enhance the interaction of the microslide with samples that are deposited onto its surface.

The microslide substrate described in the present invention has the advantage of allowing direct imaging onto a CCD camera faceplate, minimizing the need for costly optics often associated with a microscope or microarray reader. A FOI microslide of the invention can also itself serve as the faceplate of a CCD camera. CCD cameras with integral fiber optic bundle tapers can image over large area making it possible to directly simultaneously interrogate microslides that are the size of standard microscope slides or larger, or microtiter plates having fiberoptical microslide glass bottoms.

In one embodiment the invention provides an imaging device coupled with a microslide substrate. The fiber optical components of a microslide substrate of the invention provide a plurality of light trapping optical micro channels between the first and second surfaces of the substrate, and provide a viewing plane translation for an attached imaging device. A specimen on one surface of the substrate has its optical nature translated to the other surface of the substrate, which interfaces with, or is integrated into, the imaging device.

When a luminescent reaction occurs on the surface of the microslide, the light travels through the fibers of the microslide to the opposite surface. Mechanical fixtures can be used to press the microslide bottom so that it buts directly to the outer surface of the CCD camera faceplate. Light travels from the surface of the microslide through the camera faceplate and impinges directly onto the CCD sensor.

Fluorescent reactions can be monitored in a similar way. For example, an appropriately tagged sample applied to the surface of a microslide is exposed to an excitation wavelength. This can be accomplished by a number of strategies. For example, the excitation wavelength can come from various white light sources appropriately filtered to isolate the excitation wavelengths of interest. Lasers, LED's or laser diodes can also be used. A variety of strategies can be used to direct the excitation light to the surface of the microarray, including, for example, fiber optic light guides such as described in U.S. Pat. No. 6,620,623, which is hereby incorporated by reference. Upon exposure to the excitation light, the sample emits a fluorescent signal. In order to enhance the sensitivity for measurement, it may be desirable to isolate the emitted signal from the excitation signal. This can be accomplished by coating the bottom surface of the microslide with a multiplier dichroic filter designed to block certain wavelengths, while passing other wavelengths. Alternatively, if the CCD camera is intended for dedicated fluorescence measurements, the dichroic filter may be applied onto the surface of the fiber optic taper camera faceplate and, optionally, can become a permanent part of the camera. Still other strategies can be employed that take advantage of the optical properties of the fiber optical microslide. For example, the sample can be exposed to the excitation wavelength at angles, based on the NA of the microslide, selected to enable excitation of the sample while insuring that it is not transmitted through the microslide.

Functional Surface Treatments and Coatings for Microarraying Applications with Microslides of the Invention

Various surface treatments and coatings can be employed to optimize the use of an embodiment of the invention for microarraying applications. These applications include, but are not limited to: gene expression monitoring, mutation detection and analysis, genotyping of eukaryotes, microorganisms, and viruses, mapping of genomes and clones, protein detection and quantification, functional protein and peptide assays and cell/tissue microarrays. The following surface treatment and coatings are examples contemplated by the invention:

Optically flat. Microslides can be ground and polished to tight tolerances eliminating intra-slide thickness deviation, and inter-slide thickness variability. Furthermore, since microslides are not subject to thickness related optical effects, the slide is generally not subject to the type of concave or convex warp that affects conventional microscope slides that are manufactured very thin (150 microns, for example) to minimize thickness related optical effects.

Pre-cleaned. Microslide substrates can be offered with various levels of cleanliness and packaging uniquely suited for microarraying applications, such as those listed below:
Uncleaned. Microlides can be sold as manufactured, with cleaning left to the user according to their needs,
Ultrasonically cleaned. Ultrasonic cleaning procedure is used to remove all particles, debris and surface contaminants that might result from microslide manufacture.
Plasma Cleaned. Sputter etching (Ar) and reactive ion etching (RIE) (O2, CF4) allows cleaning and activation of substrate surfaces for optimum adhesion.
Cleanroom cleaned. Following ultrasonic cleaning, microslides can be sealed in a protective foil pouch under inert atmosphere in a class 100 cleanroom environment. Specialty packaging can be employed to protect the microslide from breakage, external contamination, as well as to isolate the glass surface and any special coatings from the effects of light and humidity.

Passivating surface. For certain applications, it can be advantageous for the microslide surface to have a uniform chemical composition rather than one in which the composition varies depending on whether the underlying glass is core glass, clad glass, or EMA glass. It can also be advantageous for the surface of the microslide to have a passive composition rather than one characterized by the some of the glass components characteristic of core, clad or EMA glass. Thin, well adhered, transparent coatings can be applied to the surface of the microslide by a variety of deposition techniques including (but not limited to) vacuum evaporation, sputtering, laser ablation, reactive ion platting, pinhole-free plasma enhanced deposition, organo-metallic dip coatings, spray techniques, etc. For example, a uniform, conformal, pinhole-free, well adhered coating of silicon dioxide (SiO₂) can be applied to the surface of the microslide by reactive ion platting.

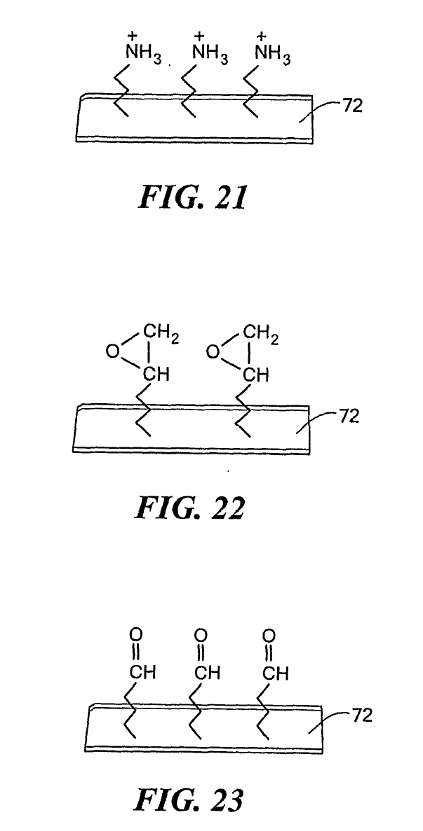

Amine DNA coupling layer. Various multifunctional aminosilane coatings can be used to coat the surface of the microslide. These coatings can enhance electrostatic attraction and provide improved binding and immobilization of cDNA molecules and PCR products. FIG. 21 depicts an amine coated surface.

Epoxy coupling layer. Epoxy coatings, such as depicted in FIG. 22, can enhance the surface of the microslide for covalent immobilization of amino-modified and unmodified oligonucleotides. Oligonucleotides are short nucleic acids (DNA or RNA) that are polymers of two to about one hundred nucleotides; longer nucleic acids are polynucleotides. The nucleic acids react with the epoxy such asmodified surface to form a stable covalent bond.

Aldehyde group coupling layer. Aldehyde coatings, such as depicted in FIG. 23, can enhance the surface of the microslide for covalent immobilization of amino-modified nucleic acids or small protein fragments such as peptides.

Permeable, 3D hydrogel coatings. These coatings can enhance the covalent immobilization of peptides, and proteins, such as antibodies, antibody fragments, enzymes or receptors. The 3D hydrogel coating preserves the three-dimensional structure of the samples being immobilized.

Other coatings, including coatings used to alter hydrophobic or hydrophilic characteristics, can be applied to the surface of the microslide.

Coated microslides may be shaped in a way that insures that the microarray is only deposited (spotted) onto the coated side. For example, one corner of the slide can be notched so that it only fits into the slide holder with the coated side facing up for microarray deposition.

The microslides described in this invention can be laser scribed with an identifying bar code, product ID, corporate logo or other identifying information. This type of barcode can be read with common microarray scanners and it is robust enough to withstand standard microarray hybridization and washing procedures.

### Fully Integrated Microslide Kits of the Invention for Microarraying and Other Applications

The embodiments of the invention can be integrated as part of a kit that includes one or more microslides, solutions and hardware to deposit microarray samples onto the microslide, labeling dyes, reagents for analysis of the microarray, and software for analysis of the results. Solutions and reagents can be provided that cover key microarray process steps: spotting, blocking, hybridization, and washing.

Standardized pre-mixed buffers and solutions can also be used to improve spot deposition during the formation of a microarray. These solutions generally reduce preparation time and run-to-run variability and can enhance spot morphology while reducing non-specific background.

Microarray reactions can also be commonly tracked using fluorescent-labeled proteins and DNA molecules. These dyes can be offered as an integral kit component of or as an embodiment of the invention. A variety of fluorescent dyes are available commercially, such as Cy3 and Cy5 (Amersham Biosciences) or Alex Fluor 647 and Alex Fluor 555 (Molecular Probes). Other dyes can be custom fabricated such as to ensure the following features: strong absorption, high fluorescence quantum yield, high photo stability, good water solubility, and increased intensity when coupled to biomolecules (thus reducing the influence of uncoupled dye).

The examples herein are provided to illustrate advantages of the present invention that have not been previously described and to further assist a person of ordinary skill within the art with fabrication of a microslide device according to the invention. The examples can include or incorporate any of the variations or embodiments of the invention described above. Moreover, the embodiments described above may each include or incorporate the variations of any or all other embodiments of the invention. The examples that follow are not intended in any way to otherwise limit the scope of the disclosure.

### Example I

### Microarray Analysis of Proteins

In this example, immunoglobulin (antibody) samples are analyzed for their binding affinity to a fluorescently labeled antigen. First, the immunoglobulin samples are diluted into a printing buffer at 0.1-0.5 μg/μl. Then, immunoglobulin samples are printed as a microarray onto an epoxy treated microslide using a split pen printing device. A blocking solution is then used to neutralize unreacted epoxy groups on the surface of the microslide. The processed microarrays are reacted with a solution containing a fluorescent labeled antigen, and allowed to incubate to achieve binding equilibrium. The microarray is then washed to remove unreacted fluorescent material. The microarray is imaged by placing it directly onto the faceplate of a CCD camera to produce an image of fluorescence on the microarray. The fluorescence data are then analyzed to evaluate the relative binding affinity of the immunoglobulins in the microarray for the antigen.

### Example II

### Microarray Analysis of DNA

This example illustrates the use of a microarray formed on a microslide of the invention to sequence a genomic DNA from a bacterium. A series of DNA samples containing fragments representative of the genomic DNA of a bacterium are diluted into a spotting solution. The DNA samples are printed as a microarray on the epoxy coated surface of a microslide substrate. Unreacted epoxy groups are blocked. The fluorescently labeled sample of bacterial genomic DNA for sequence analysis is hybridized to the samples in the microarray. Unbound label is washed away. The microslide is scanned in a laser scanning microarray reader, and the fluorescence at each spot in the microarray is determined, thereby allowing the determination of the nucleotide sequence of the sample to be determined in a computer.

While the present invention has been described herein in conjunction with a preferred embodiment, a person with ordinary skill in the art, after reading the foregoing, can effect changes, substitutions of equivalents and other types of alterations to the invention as set forth herein. Each embodiment described herein can also have included or incorporated therewith such variations as disclosed in regard to any or all of the other embodiments.

What is claimed is:

1. A fiber optic interrogated microslide having zero thickness optical equivalence, the microslide comprising:
a substrate comprising an upper surface and a lower surface; and
a plurality of optic fibers integrally disposed in the substrate, plural optic fibers optically coupling the upper and lower surfaces of the substrate, wherein optically coupling the upper and lower surfaces of the substrate provides for substantially zero thickness optical interrogation.

2. The microslide of claim 1, wherein the upper and lower surfaces of the substrate are substantially parallel.

3. The microslide of claim 1, wherein the fibers are essentially parallel to one another.

4. The microslide of claim 1, wherein the fibers are essentially normal to the upper and lower surfaces of the substrate.

5. The microslide of claim 1, wherein the fibers form a taper or an inverter.

6. The microslide of claim 1, further comprising a chemically modified surface of the substrate.

7. The microslide of claim 6, wherein the chemical modification is the covalent addition of an amino group, an epoxy group, or an aldehyde group.

8. The microslide of claim 7, further comprising a covalently bound peptide, polypeptide, oligonucleotide, or polynucleotide.

9. The microslide of claim 8, wherein the polypeptide is an immunoglobulin or fragment thereof.

10. The microslide of claim 8 comprising an array of covalently bound oligonucleotides or polynucleotides having sequences representative of genomic DNA isolated from a cell or organism.

11. The microslide of claim 8 comprising an array of covalently bound oligonucleotides or polynucleotides suitable for use in nucleic acid sequencing.

12. The microslide of claim 1, wherein at least one surface of the substrate is passivated.

13. The microslide of claim 12, wherein the passivation is by a coating deposited by vacuum evaporation, sputtering, laser ablation, reactive ion platting, plasma enhanced deposition, organo-metallic dipping, spraying or a combination thereof.

14. A system for zero thickness optical interrogation of a sample, the system comprising:
the fiber optic interrogated microslide of claim 1;
a sample disposed on a surface of the substrate of said microslide, whereby a means for viewing the sample from an opposite surface experiences optical coupling of the surfaces of the substrate providing for substantially zero thickness optical interrogation of the sample.

15. The system of claim 14, further comprising an imaging device for viewing the sample.

16. The system of claim 15, wherein the imaging device is selected from the group consisting of a microscope, a camera, and a sensor array.

17. The system of claim 16, wherein the microslide serves as the faceplate for a charge coupled device camera.

18. The system of claim 17, wherein the microslide is a taper or an inverter.

19. The system of claim 16, wherein the microslide forms the base of a cell culture vessel suitable for use with an inverted microscope.

20. The system of claim 14, wherein the microslide provides for greater light collection efficiency to optically interrogate the sample compared to using a plate glass substrate.

21. The system of claim 14, wherein the microslide provides for greater resolution to optically interrogate the sample compared to using a plate glass substrate.

22. The system of claim 14, wherein the microslide provides for less chromatic dispersion to optically interrogate the sample compared to using a plate glass substrate.

23. The system of claim 14, capable of monitoring a chemical reaction in the sample.

24. The system of claim 23, wherein the chemical reaction results in chemiluminescence.

25. The system of claim 23, wherein the chemical reaction results in binding of a labeled moiety to the substrate.

26. The system of claim 25, wherein the labeled moiety is fluorescent, radioactive, or possesses enzyme activity.

27. The system of claim 14, further comprising a split pen printing device.

28. A kit comprising:
the microslide of claim 1; and
a reagent to be disposed on a surface of the substrate.

29. The kit of claim 28, wherein the reagent comprises one or more of pharmaceutical compounds, genomic components, metallic components, metals, polymeric components, polymers, polyether, ether, ketones, polyimides, epoxies, nylons, homopolymers, heteropolymers, polycarbonates, glass, acetal polymers, acrylate polymers, methacrylate polymers, copolymers, terpolymers, cellulosic polymers, cellulose acetates, cellulose nitrates, cellulose propionates, cellulose acetate butyrates, cellophanes, rayons, rayon triacetates, cellulose ethers, carboxymethyl celluloses, hydroxyalkyl celluloses, polyoxymethylene polymers, polyimide polymers, polyether block imides, polybismaleinimides, polyamidimides, polyesterimides, polyetherimides, polysulfone polymers, polyarylsulfones, polyethersulfones, polyamide polymers, nylon 6,6, polycaprolactams, polyacrylamides, resins, alkyd resins, phenolic resins, urea resins, melamine resins, epoxy resins, allyl resins, epoxide resins, polycarbonates, polyacrylonitriles, polyvinylpyrrolidones, anhydride polymers, maleic anhydride polymers, polymers of vinyl monomers, polyvinyl alcohols, polyvinyl halides, polyvinyl chlorides, ethylene vinylacetate copolymers, polyvinylidene chlorides, polyvinyl ethers, polyvinyl methyl ethers, polystyrenes, styrene butadiene copolymers, acrylonitrile styrene copolymers, acrylonitrile butadiene styrene copolymers, styrene butadiene styrene copolymers, styrene isobutylene styrene copolymers, polyvinyl ketones, polyvinylcarbazoles, polyvinyl esters, polyvinyl acetates, hydrogels, polybenzimidazoles, ionomers, polyalkyl oxide polymers, polyethylene oxides, glycosaminoglycans, polyesters, polyethylene terephthalates, aliphatic polyesters, polymers of lactide, epsilon caprolactone, glycolide, glycolic acid, hydroxybutyrate, hydroxyvalerate, paradioxanone, trimethylene carbonate, 1,4-dioxepan-2-one, 1,5-dioxepan-2-one, 6,6-dimethyl-1,4-dioxan-2-one, polyether polymers, polyarylethers, polyphenylene ethers, polyether ketones, polyether ether ketones, polyphenylene sulfides, polyisocyanates, polyolefin polymers, polyalkylenes, polypropylenes, polyethylenes, polybutylenes, polybut-1-ene, polyisobutylene, poly-4-methyl-pen-1-enes, ethylene-alpha-olefin copolymers, ethylene-methyl methacrylate copolymers, ethylene-vinyl acetate copolymers, fluorinated polymers, polytetrafluoroethylenes, poly(tetrafluoroethylene-co-hexafluoropropene), modified ethylene-tetrafluoroethylene copolymers, polyvinylidene fluorides, silicone polymers, polyurethanes, polyurethane dispersions, p-xylylene polymers, polyiminocarbonates, copoly(ether-esters), polyethylene oxide-polylactic acid copolymers, polyphosphazines, polyalkylene oxalates, polyoxaamides, polyoxaesters, amines, amino groups, polyorthoesters, biopolymers, polypeptides, proteins, polysaccharides, fatty acids, esters of fatty acids, fibrin, fibrinogen, collagen, elastin, chitosan, gelatin, starch, glycosaminoglycans, hyaluronic acid, therapeutic agents, oligonucleotides, proteins, antisense polynucleotides, polynucleotides coding for a specific product, genetic recombinant components, nucleic acids, DNA, cDNA, mRNA, tRNA, RNA, polynucleotides, viruses, bacteria, phage, histones, non-infectious vectors, vectors, plasmids, lipids, liposomes, cationic polymers, cationic lipids, viral vectors, virus-like particles, synthetic virus particles, peptide targeting sequences, antisense nucleic acids, genomic sequences, DNA chimeras, gene sequences encoding for ferry proteins, membrane translocating sequences, cells, ribozymes, antisense oligonucleotides, DNA compacting agents, gene or vector systems, polynucleotides, recombinant nucleic acids, naked DNA, cDNA, mRNA, tRNA or RNA, genomic DNA, cDNA, mRNA, tRNA or RNA in a non-infectious vector or in a viral vector, human origin cells, autologous cells, allogeneic cells, animal source cells, xenogeneic cells, genetically engineered proteins, polymerized chain reaction components, blood, serums, bodily fluids, tissues or any combination thereof.

30. A kit comprising:
the microslide of claim 1; and
a functional agent for coating at least one surface of the substrate of the microslide.

31. The kit of claim 12, wherein the functional agent comprises an aminosilane, epoxy, aldehyde or combinations thereof.

32. The kit of claim 31, wherein the functional agent comprises one or more of pharmaceutical compounds, genomic components, metallic components, metals, polymeric components, polymers, polyether, ether, ketones, polyimides, epoxies, nylons, homopolymers, heteropolymers, polycarbonates, glass, acetal polymers, acrylate polymers, methacrylate polymers, copolymers, terpolymers, cellulosic polymers, cellulose acetates, cellulose nitrates, cellulose propionates, cellulose acetate butyrates, cellophanes, rayons, rayon triacetates, cellulose ethers, carboxymethyl celluloses, hydroxyalkyl celluloses, polyoxymethylene polymers, polyimide polymers, polyether block imides, polybismaleinimides, polyamidimides, polyesterimides, polyetherimides, polysulfone polymers, polyarylsulfones, polyethersulfones, polyamide polymers, nylon 6,6, polycaprolactams, polyacrylamides, resins, alkyd resins, phenolic resins, urea resins, melamine resins, epoxy resins, allyl resins, epoxide resins, polycarbonates, polyacrylonitriles, polyvinylpyrrolidones, anhydride polymers, maleic anhydride polymers, polymers of vinyl monomers, polyvinyl alcohols, polyvinyl halides, polyvinyl chlorides, ethylene vinylacetate copolymers, polyvinylidene chlorides, polyvinyl ethers, polyvinyl methyl ethers, polystyrenes, styrene butadiene copolymers, acrylonitrile styrene copolymers, acrylonitrile butadiene styrene copolymers, styrene butadiene styrene copolymers, styrene isobutylene styrene copolymers, polyvinyl ketones, polyvinylcarbazoles, polyvinyl esters, polyvinyl acetates, hydrogels, polybenzimidazoles, ionomers, polyalkyl oxide polymers, polyethylene oxides, glycosaminoglycans, polyesters, polyethylene terephthalates, aliphatic polyesters, polymers of lactide, epsilon caprolactone, glycolide, glycolic acid, hydroxybutyrate, hydroxyvalerate, paradioxanone, trimethylene carbonate, 1,4-dioxepan-2-one, 1,5-dioxepan-2-one, 6,6-dimethyl-1,4-dioxan-2-one, polyether polymers, polyarylethers, polyphenylene ethers, polyether ketones, polyether ether ketones, polyphenylene sulfides, polyisocyanates, polyolefin polymers, polyalkylenes, polypropylenes, polyethylenes, polybutylenes, polybut-1-ene, polyisobutylene, poly-4-methyl-pen-1-enes, ethylene-alpha-olefin copolymers, ethylene-methyl methacrylate copolymers, ethylene-vinyl acetate copolymers, fluorinated polymers, polytetrafluoroethylenes, poly(tetrafluoroethylene-co-hexafluoropropene), modified ethylene-tetrafluoroethylene copolymers, polyvinylidene fluorides, silicone polymers, polyurethanes, polyurethane dispersions, p-xylylene polymers, polyiminocarbonates, copoly(ether-esters), polyethylene oxide-polylactic acid copolymers, polyphosphazines, polyalkylene oxalates, polyoxaamides, polyoxaesters, amines, amino groups, polyorthoesters, biopolymers, polypeptides, proteins, polysaccharides, fatty acids, esters of fatty acids, fibrin, fibrinogen, collagen, elastin, chitosan, gelatin, starch, glycosaminoglycans, hyaluronic acid, therapeutic agents, oligonucleotides, proteins, antisense polynucleotides, polynucleotides coding for a specific product, genetic recombinant components, nucleic acids, DNA, cDNA, mRNA, tRNA, RNA, polynucleotides, viruses, bacteria, phage, histones, non-infectious vectors, vectors, plasmids, lipids, liposomes, cationic polymers, cationic lipids, viral vectors, virus-like particles, synthetic virus particles, peptide targeting sequences, antisense nucleic acids, genomic sequences, DNA chimeras, gene sequences encoding for ferry proteins, membrane translocating sequences, cells, ribozymes, antisense oligonucleotides, DNA compacting agents, gene or vector systems, polynucleotides, recombinant nucleic acids, naked DNA, cDNA, mRNA, tRNA or RNA, genomic DNA, cDNA, mRNA, tRNA or RNA in a non-infectious vector or in a viral vector, human origin cells, autologous cells, allogeneic cells, animal source cells, xenogeneic cells, genetically engineered proteins, polymerized chain reaction components, blood, serums, bodily fluids, tissues or any combination thereof.

33. A method for zero thickness optical interrogation of a sample, the method comprising:
providing the microslide of claim 1 and said sample;
disposing the sample on a surface of the substrate of the microslide; and
optically interrogating the sample from an opposite surface of the substrate.

34. A method for zero thickness optical interrogation of an event, the method comprising:
providing the system of claim 15;
initiating an event in the sample disposed on a surface of the substrate; and
optically interrogating the event in the sample with the imaging device from an opposite side of the substrate.

35. The method of claim 34, wherein the imaging device is selected from the group consisting of a microscope, camera, and a sensor array.

36. The method of claim 33, wherein the step of interrogating from a surface of the substrate includes interrogating from a surface of a microtiter plate, a microscope slide, a microarray plate or a combination thereof.

37. The method of claim 33 further comprising coating one or both surfaces of the substrate of the microslide with a functional agent.

38. The method of claim 37, wherein the functional agent comprises an aminosilane, epoxy, aldehyde or a combination thereof.

39. The method of claim 37, wherein the functional agent comprises one or more of pharmaceutical compounds, genomic components, metallic components, metals, polymeric components, polymers, polyether, ether, ketones, polyimides, epoxies, nylons, homopolymers, heteropolymers, polycarbonates, glass, acetal polymers, acrylate polymers, methacrylate polymers, copolymers, terpolymers, cellulosic polymers, cellulose acetates, cellulose nitrates, cellulose propionates, cellulose acetate butyrates, cellophanes, rayons, rayon triacetates, cellulose ethers, carboxymethyl celluloses, hydroxyalkyl celluloses, polyoxymethylene polymers, polyimide polymers, polyether block imides, polybismaleinimides, polyamidimides, polyesterimides, polyetherimides, polysulfone polymers, polyarylsulfones, polyethersulfones, polyamide polymers, nylon 6,6, polycaprolactams, polyacrylamides, resins, alkyd resins, phenolic resins, urea resins, melamine resins, epoxy resins, allyl resins, epoxide resins, polycarbonates, polyacrylonitriles, polyvinylpyrrolidones, anhydride polymers, maleic anhydride polymers, polymers of vinyl monomers, polyvinyl alcohols, polyvinyl halides, polyvinyl chlorides, ethylene vinylacetate copolymers, polyvinylidene chlorides, polyvinyl ethers, polyvinyl methyl ethers, polystyrenes, styrene butadiene copolymers, acrylonitrile styrene copolymers, acrylonitrile butadiene styrene copolymers, styrene butadiene styrene copolymers, styrene isobutylene styrene copolymers, polyvinyl ketones, polyvinylcarbazoles, polyvinyl esters, polyvinyl acetates, hydrogels, polybenzimidazoles, ionomers, polyalkyl oxide polymers, polyethylene oxides, glycosaminoglycans, polyesters, polyethylene terephthalates, aliphatic polyesters, polymers of lactide, epsilon caprolactone, glycolide, glycolic acid, hydroxybutyrate, hydroxyvalerate, paradioxanone, trimethylene carbonate, 1,4-dioxepan-2-one, 1,5-dioxepan-2-one, 6,6-dimethyl-1,4-dioxan-2-one, polyether polymers, polyarylethers, polyphenylene ethers, polyether ketones, polyether ether ketones, polyphenylene sulfides, polyisocyanates, polyolefin polymers, polyalkylenes, polypropylenes, polyethylenes, polybutylenes, polybut-1-ene, polyisobutylene, poly-4-methyl-pen-1-enes, ethylene-alpha-olefin copolymers, ethylene-methyl methacrylate copolymers, ethylene-vinyl acetate copolymers, fluorinated polymers, polytetrafluoroethylenes, poly(tetrafluoroethylene-co-hexafluoropropene), modified ethylene-tetrafluoroethylene copolymers, polyvinylidene fluorides, silicone polymers, polyurethanes, polyurethane dispersions, p-xylylene polymers, polyiminocarbonates, copoly(ether-esters), polyethylene oxide-polylactic acid copolymers, polyphosphazines, polyalkylene oxalates, polyoxaamides, polyoxaesters, amines, amino groups, polyorthoesters, biopolymers, polypeptides, proteins, polysaccharides, fatty acids, esters of fatty acids, fibrin, fibrinogen, collagen, elastin, chitosan, gelatin, starch, glycosaminoglycans, hyaluronic acid, therapeutic agents, oligonucleotides, proteins, antisense polynucleotides, polynucleotides coding for a specific product, genetic recombinant components, nucleic acids, DNA, cDNA, mRNA, tRNA, RNA, polynucleotides, viruses, bacteria, phage, histones, non-infectious vectors, vectors, plasmids, lipids, liposomes, cationic polymers, cationic lipids, viral vectors, virus-like particles, synthetic virus particles, peptide targeting sequences, antisense nucleic acids, genomic sequences, DNA chimeras, gene sequences encoding for ferry proteins, membrane translocating sequences, cells, ribozymes, antisense oligonucleotides, DNA compacting agents, gene or vector systems, polynucleotides, recombinant nucleic acids, naked DNA, cDNA, mRNA, tRNA or RNA, genomic DNA, cDNA, mRNA, tRNA or RNA in a non-infectious vector or in a viral vector, human origin cells, autologous cells, allogeneic cells, animal source cells, xenogeneic cells, genetically engineered proteins, polymerized chain reaction components, blood, serums, bodily fluids, tissues or any combination thereof.

40. The method of claim 37 further comprising immobilizing a material on a surface of the substrate using the functional agent.

41. The method of claim 40, wherein the material comprises a compound of biological origin, a synthetic compound, a metallic compound or a combination thereof.

42. The method of claim 41, wherein the material comprises one or more of pharmaceutical compounds, genomic components, metallic-components, metals, polymeric components, polymers, polyether, ether, ketones, polyimides, epoxies, nylons, homopolymers, heteropolymers, polycarbonates, glass, acetal polymers, acrylate polymers, methacrylate polymers, copolymers, terpolymers, cellulosic polymers, cellulose acetates, cellulose nitrates, cellulose propionates, cellulose acetate butyrates, cellophanes, rayons, rayon triacetates, cellulose ethers, carboxymethyl celluloses, hydroxyalkyl celluloses, polyoxymethylene polymers, polyimide polymers, polyether block imides, polybismaleinimides, polyamidimides, polyesterimides, polyetherimides, polysulfone polymers, polyarylsulfones, polyethersulfones, polyamide polymers, nylon 6,6, polycaprolactams, polyacrylamides, resins, alkyd resins, phenolic resins, urea resins, melamine resins, epoxy resins, allyl resins, epoxide resins, polycarbonates, polyacrylonitriles, polyvinylpyrrolidones, anhydride polymers, maleic anhydride polymers, polymers of vinyl monomers, polyvinyl alcohols, polyvinyl halides, polyvinyl chlorides, ethylene vinylacetate copolymers, polyvinylidene chlorides, polyvinyl ethers, polyvinyl methyl ethers, polystyrenes, styrene butadiene copolymers, acrylonitrile styrene copolymers, acrylonitrile butadiene styrene copolymers, styrene butadiene styrene copolymers, styrene isobutylene styrene copolymers, polyvinyl ketones, polyvinylcarbazoles, polyvinyl esters, polyvinyl acetates, hydrogels, polybenzimidazoles, ionomers, polyalkyl oxide polymers, polyethylene oxides, glycosaminoglycans, polyesters, polyethylene terephthalates, aliphatic polyesters, polymers of lactide, epsilon caprolactone, glycolide, glycolic acid, hydroxybutyrate, hydroxyvalerate, paradioxanone, trimethylene carbonate, 1,4-dioxepan-2-one, 1,5-dioxepan-2-one, 6,6-dimethyl-1,4-dioxan-2-one, polyether polymers, polyarylethers, polyphenylene ethers, polyether ketones, polyether ether ketones, polyphenylene sulfides, polyisocyanates, polyolefin polymers, polyalkylenes, polypropylenes, polyethylenes, polybutylenes, polybut-1-ene, polyisobutylene, poly-4-methyl-pen-1-enes, ethylene-alpha-olefin copolymers, ethylene-methyl methacrylate copolymers, ethylene-vinyl acetate copolymers, fluorinated polymers, polytetrafluoroethylenes, poly(tetrafluoroethylene-co-hexafluoropropene), modified ethylene-tetrafluoroethylene copolymers, polyvinylidene fluorides, silicone polymers, polyurethanes, polyurethane dispersions, p-xylylene polymers, polyiminocarbonates, copoly(ether-esters), polyethylene oxide-polylactic acid copolymers, polyphosphazines, polyalkylene oxalates, polyoxaamides, polyoxaesters, amines, amino groups, polyorthoesters, biopolymers, polypeptides, proteins, polysaccharides, fatty acids, esters of fatty acids, fibrin, fibrinogen, collagen, elastin, chitosan, gelatin, starch, glycosaminoglycans, hyaluronic acid, therapeutic agents, oligonucleotides, proteins, antisense polynucleotides, polynucleotides coding for a specific product, genetic recombinant components, nucleic acids, DNA, cDNA, mRNA, tRNA, RNA, polynucleotides, viruses, bacteria, phage, histones, non-infectious vectors, vectors, plasmids, lipids, liposomes, cationic polymers, cationic lipids, viral vectors, virus-like particles, synthetic virus particles, peptide targeting sequences, antisense nucleic acids, genomic sequences, DNA chimeras, gene sequences encoding for ferry proteins, membrane translocating sequences, cells, ribozymes, antisense oligonucleotides, DNA compacting agents, gene or vector systems, polynucleotides, recombinant nucleic acids, naked DNA, cDNA, mRNA, tRNA or RNA, genomic DNA, cDNA, mRNA, tRNA or RNA in a non-infectious vector or in a viral vector, human origin cells, autologous cells, allogeneic cells, animal source cells, xenogeneic cells, genetically engineered proteins, polymerized chain reaction components, blood, serums, bodily fluids, tissues or any combination thereof.

43. The method of claim 42, wherein immobilization is through covalent bonding, chemical interaction, physical interaction, electrostatic interaction, mechanical interaction, hybridization or a combination thereof.

44. The method of claim 37, including the step of forming a microarray on a surface of the substrate.

45. The method of claim 44, including forming the microarray by split pin printing, spotting or a combination thereof.

46. The method of claim 34, wherein said optical interrogation provides one or more of gene expression monitoring, mutation detection, mutation analysis, genotyping, genomic mapping, clone mapping, protein detection, protein quantification, protein expression monitoring, assaying enzyme activity, assaying receptor binding, or any combination thereof.

47. An imaging device having a specimen supporting substrate, the substrate providing spatial translation of a viewing plane of the imaging device, the substrate comprising:
first and second surfaces, one of which is conformal to the viewing plane;
a plurality of optical micro channels between the first and second surface providing said viewing plane translation;
the other of said surfaces providing support for the specimen.

48. The imaging device of claim 47 wherein said micro channels comprise optical fibers.

49. A specimen supporting substrate, the substrate providing spatial translation of a viewing plane of an imaging device, the substrate comprising:
first and second surfaces, one of which is conformal to the viewing plane;
a plurality of optical micro channels between the first and second surface providing said viewing plane translation.

50. The substrate of claim 49 wherein said micro channels comprise optical fibers.

51. A specimen supporting substrate, the substrate providing spatial translation of a viewing plane, the substrate comprising:
first and second surfaces, one of which is conformal to the viewing plane;
a plurality of optical micro channels between the viewing plane conformal surface and the other surface providing said viewing plane translation there between;
a sensor array on the surface conformal to the viewing plane; and
the other of said surfaces providing support for the specimen.

52. The substrate of claim 51 wherein said substrate comprises physically separable elements capable of being placed adjacent to each other, each having a separate plurality of optical micro channels which when said elements are adjacent provide a single said plurality of optical micro channels.

53. The substrate of claim 51 or 52 wherein said micro channels comprise optical fibers.
